SEQUENCE LISTING (47 sequences)

SEQ ID NO: 1 - DNA, 33 bp, Homo sapiens
cagatggaaaccaacasgcatttgctgtgggct

SEQ ID NO: 2 - DNA, 33 bp, Homo sapiens
ctactcagcagaatacragcagaggtcagagaa

SEQ ID NO: 3 - DNA, 33 bp, Homo sapiens
ctttattgaggtataawtgctatgcaaaaaaaa

SEQ ID NO: 4 - DNA, 33 bp, Homo sapiens
gctcaggagctattagrattttttttgtatcga

SEQ ID NO: 5 - DNA, 33 bp, Homo sapiens
ataacagtatcaggacragcttctctaggaggg

SEQ ID NO: 6 - DNA, 33 bp, Homo sapiens
aacaaatacctgttaartatcttttgggtgcca

SEQ ID NO: 7 - DNA, 33 bp, Homo sapiens
tgtgcctattgacttgyactatgcacgacctat

SEQ ID NO: 8 - DNA, 33 bp, Homo sapiens
agaggcagaatcaccartgactcataccccttg

SEQ ID NO: 9 - DNA, 33 bp, Homo sapiens
cagagctaccctgctgraggagttaggtatgca

SEQ ID NO: 10 - DNA, 33 bp, Homo sapiens
catatctatgcttccaygctcatggtatataag

SEQ ID NO: 11 - DNA, 33 bp, Homo sapiens
ccatccacatcccaaakcactgacagtctaact

SEQ ID NO: 12 - DNA, 33 bp, Homo sapiens
agtcaacctaaaataarattggcaacagttaaa

SEQ ID NO: 13 - DNA, 33 bp, Homo sapiens
agtgggtatccattaaygaatgcatctgttcat

SEQ ID NO: 14 - DNA, 33 bp, Homo sapiens
tcatgctggtgtttcargtttctgacattgctg

SEQ ID NO: 15 - DNA, 33 bp, Homo sapiens
acgatgaaggctatgayctccagcctacgtttt

SEQ ID NO: 16 - DNA, 33 bp, Homo sapiens
aacatcttttaatatcmctgttggtcacttgta

SEQ ID NO: 17 - DNA, 33 bp, Homo sapiens
ggacacctggcttctarttcacccacactgcat

SEQ ID NO: 18 - DNA, 33 bp, Homo sapiens
actacagtgatcaaaayagtctactggcataag

SEQ ID NO: 19 - DNA, 33 bp, Homo sapiens
taaaatctaattttaarccaacatatccatgtg

SEQ ID NO: 20 - DNA, 33 bp, Homo sapiens
cggaaatctaaatctgyacctagatcctgtgct

SEQ ID NO: 21 - DNA, 33 bp, Homo sapiens
tacttttataagcccargaaagatcccaaccca

SEQ ID NO: 22 - DNA, 33 bp, Homo sapiens
ttaattgtcttataaarttgtcactcatttgga

SEQ ID NO: 23 - DNA, 33 bp, Homo sapiens
gatggataatccaccayaacgagactgattttt

SEQ ID NO: 24 - DNA, 33 bp, Homo sapiens
tcacctatatcctggarcggttgtgaggataaa

SEQ ID NO: 25 - DNA, 33 bp, Homo sapiens
cccccactagagaccamgcatctggaaatggtg

SEQ ID NO: 26 - DNA, 33 bp, Homo sapiens
ctgagataattccttakaagtctgagtagggaa

SEQ ID NO: 27 - DNA, 33 bp, Homo sapiens
ttaattacagttttgawgttagtgatgtttgtc

SEQ ID NO: 28 - DNA, 33 bp, Homo sapiens
ctccttctgacactggyaaaactgtgaaggtgt

SEQ ID NO: 29 - DNA, 33 bp, Homo sapiens
gtgggtctaaacacaaygagatggaaggagaca

SEQ ID NO: 30 - DNA, 33 bp, Homo sapiens
ttagcatgtacccttayatgtcattggccaagt

SEQ ID NO: 31 - DNA, 33 bp, Homo sapiens
accatgtgacaactgamagatgccatgttaagc

SEQ ID NO: 32 - DNA, 33 bp, Homo sapiens
ttcatagattcacagargggaagatagccatgc

SEQ ID NO: 33 - DNA, 33 bp, Homo sapiens
accgtccttgagattcyattggatttacttcca

SEQ ID NO: 34 - DNA, 33 bp, Homo sapiens
aatgcttagcaatgaawgcttaaatgagaaact

SEQ ID NO: 35 - DNA, 33 bp, Homo sapiens
tacaggtattatgcttmaaaaaaggttttcttt

SEQ ID NO: 36 - DNA, 33 bp, Homo sapiens
tgaactggaccttgaargataatcattttctag

SEQ ID NO: 37 - DNA, 33 bp, Homo sapiens
caaagtgtatactcaakgactaattaggatgct

SEQ ID NO: 38 - DNA, 33 bp, Homo sapiens
tactacccccagattarcagcctagcaggtact

SEQ ID NO: 39 - DNA, 33 bp, Homo sapiens
tctgtaaatttaactawggaaggcagtggctaa

SEQ ID NO: 40 - DNA, 33 bp, Homo sapiens
tgggaacaggaactaaytggacctttggggatg

SEQ ID NO: 41 - DNA, 33 bp, Homo sapiens
ggccaagacataccaaygaggcaggaaactatg

SEQ ID NO: 42 - DNA, 33 bp, Homo sapiens
gcttttttccagatccsaaaacccttccattca

SEQ ID NO: 43 - DNA, 33 bp, Homo sapiens
gttataaggggacaaargacggtagcttgtatt

SEQ ID NO: 44 - DNA, 33 bp, Homo sapiens
tagatcacctttggggraatcttggcactaatg

SEQ ID NO: 45 - DNA, 33 bp, Homo sapiens
agtgagtcttgacaacyaaaatggtgtgaagtg

SEQ ID NO: 46 - DNA, 33 bp, Homo sapiens
ctaaggcaatgaggacmctagagtctgcttttg

SEQ ID NO: 47 - DNA, 33 bp, Homo sapiens
catgccaacatcagcascaaagatacaaaggta

(19) United States
(12) Patent Application Publication
(10) Pub. No.: US 2015/0152498 A1
(43) Pub. Date: Jun. 4, 2015

(54) ASSAY AND METHOD FOR PREDICTING THERAPEUTIC EFFICACY OF IMMUNOGLOBULIN THERAPY IN INDIVIDUAL PATIENTS WITH RELAPSING REMITTING MULTIPLE SCLEROSIS (RR-MS)

(71) Applicants: OCTAPHARMA AG, Lachen (CH); Ruprecht-Karls-University Heidelberg, Heidelberg (DE)

(72) Inventors: Stefan Meuer, Heidelberg (DE); Thomas Giese, Heidelberg (DE); Stefan Wietek, Vienna (AT)

(21) Appl. No.: 14/406,115

(22) PCT Filed: Jun. 14, 2013
(86) PCT No.: PCT/EP2013/062355

§ 371 Date: Dec. 5, 2014

Related U.S. Application Data
(60) Provisional application No. 61/659,550, filed on Jun. 14, 2012.

(30) Foreign Application Priority Data
Jul. 27, 2012 (EP) 12178197.5

(51) Int. Cl. C12Q 1/68

(52) U.S. Cl. CPC C12Q 1/6883; C12Q 2600/106; C12Q 2600/156

(57) ABSTRACT

A method for generating a reference for determining the likelihood of response of a patient, suffering from a disease, towards immunoglobulin therapy comprising the steps of
a) providing samples of a sufficient number of individuals, in particular at least 10 individuals, the samples containing B- and T-lymphocytes, natural killer cells, invariant T-cells and monocytes of the individuals;
b) determination of values of immune parameters which are either static, such as leukocyte subpopulations and cytokine-level in the plasma or functional, like gene expression and cytokine release after lipopolysaccharide (LPS) and/or IVIG stimulation ex vivo;
c) the determined values derived from immune parameters of the samples of the individuals are ordered in quartiles and the values belonging to the 1. quartile, values distributed at the low end of the corresponding parameter, are set to “−1”, whereas values distributed in the 4. quartile are set to “+1” for an individual LDA-score calculation and values in between are set to “0”;
d) genotyping of at least two of the polynucleotides selected from the group consisting of MTM1, EIF3E, COPS8, ADAMTSL1, CXXC4, RSPO2, OR9Q1, ADAMTS9, KLHDC8A and PRDM9, in the samples of the individuals and awarding the value of 0 for specific homozygous SNP combinations (SNP—Single Nucleotide Polymorphism), which indicates that the blood sample stems from a person which will respond to immunoglobulin (IG) treatment, while awarding the value of 1 for SNP combinations not meeting that criteria, which indicates that the blood sample stems from a person which will not respond to immunoglobulin treatment;
e) combining the results of genotyping with results of immuno parameter determination in the calculation of an individual LDA-score (LDA—Linar Discriminant Analysis);
f) combining a multiplicity of individual LDA-scores to create a reference Responder Score, which allows discrimination between responders and non-responders.

The method can be employed for determining the likelihood of response of a patient, suffering from a disease, towards immunoglobulin therapy.

The invention pertains to a method of determining the individual responsiveness toward immunoglobulin therapy.

B cells identify pathogens when antibodies on their surface (B-cell receptor) bind to a specific foreign antigen. In response, B cells divide and differentiate into plasma cells, which secrete millions of copies of the antibody that recognize the activating antigen. Antibodies (also known as immunoglobulins) recognise targets comprising many different compounds, structures and those also as part of cellular structures and often neutralise their biological effect. The immune complexes are cleared fast leading to the elimination of a “target” molecule, accordingly, recognition, binding and removal function is doubtless of essential importance, which is substantiated by the fact that patients lacking (or have reduced) immunoglobulin levels are prone to serious and recurrent infections. Beyond this protection function towards intruders, immunoglobulins bear important regulatory function in balancing and regulating the immune system. Immunoglobulin products, usually derived from pooled blood or plasma donations and prepared according processes well known to the expert, are used for the treatment of IMID (immune mediated inflammatory diseases) and so called AID (autoimmune diseases), while those definitions may express identical or overlapping features of a disease. The immunoglobulin G (IgG) concentrates are usually applied intravenously (IVIG) or subcutaneously (SCIG), but may be also intramuscularly, inhaled, intra-ocularly, orally or topically. When B cells react aggressively against self, the potential for pathology is extreme. It is therefore not surprising that B-cell depletion is seen as an attractive therapy in patients with autoimmune diseases or immune mediated inflammatory diseases (IMIDs).

Natural killer (NK) cells are large granular lymphocytes that belong to the innate immune system because unlike T- or B-lymphocytes of the adaptive or antigen-specific immune system, NK cells do not rearrange T-cell receptor or immunoglobulin genes from their germline configuration. NK cells have been characterized as a lymphocyte lineage with both cytotoxic and cytokine-producing effector functions. NK cells accomplish selective lysis of cells on the basis of activating and inhibitory receptors relatively specific to the NK cell lineage. Activating receptors such as NKG2D recognize natural stress signal ligands or pathogen-derived ligands expressed on transformed or infected cells. In contrast, the inhibitory receptors of the CD94-NKG2A complex, KIR family (Killer-cell Immunoglobulin-like Receptors) bind self but not allogeneic MHC I (Major Histocompatibility Complex I). The KIR receptors are highly polymorphic, heterogeneously expressed among NK cells, and important in NK cell self-tolerance. During licensing, NK cells acquire functional competence following productive interactions between inhibitory receptors and self-MHC during development. Licensed NK cells express at least one of several possible inhibitory receptor alleles. Hence, MHC-recognition among NK cells is diverse so that at least one NK cell subset will respond to downregulation of any single MHC class I molecule. When activating NK cell effector functions are primed, IFN-γ is secreted and granzyme and perforin release are enhanced. NK cells also express Fcγ receptors, which recognize several IgG subclasses, to mediate antibody-dependent cellular cytotoxicity. These receptors and their roles in NK cell surveillance and cytolysis have been well studied and are closely associated with the identity of NK cells. Upon flow cytometry so called CD56^{bright} and CD56^{dim} can be differentiated which do or do not express Fcγ RIII (CD16). The interpretation of the flow cytometry results upon ‘gatings’ is well known to the skilled person.

Accordingly, 4 major sub-populations can be defined, which are

CD16+/CD56^{bright}

CD16+/CD56^{dim}

CD16−/CD56^{bright}

CD16−/CD56^{dim}

NK cells are an important part of the host defence. However, if dysregulated, e.g. as reason or in the course of diseases, they can direct themselves against “self-structures” with significant pathophysiological consequences, like attack of organ structures and oligodendrocytes in the periphery and brain. Although the mechanisms are not fully understood leading to such self-attacks, regaining control of these cells and reducing their killing power to a physiologically reasonable level is required. As killing efficiency is tightly connected to degranulation and release of effector molecules, it is an aim to control such degranulation to reduce damage.

The effectivity of intravenous immunoglobulin (IVIG) in autoimmune diseases was first described in the 1950ies and then in the 1980ies, when it was used to treat patients suffering from idiopathic thrombocytopenic purpura. In the meantime many clinical studies demonstrated a beneficial effect of IVIG in autoimmune diseases. Among others, IVIG therapy is proven in Guillain-Barre syndrome (GBS), Kawasaki syndrome, chronic inflammatory demyelinising polyneuropathy, myasthenia gravis and corticosteroid-resistant dermatomyositis (Ephrem et al., 2005; Kazatchkine and Kaveri, 2001; Boros et al., 2005). A positive effect of IVIG on disease progression relapse rate and MRI enhancing lesions in multiple sclerosis (MS) was demonstrated by clinical studies as well (Sorensen P S, 2003; Sorensen et al., 2002). An established method to assess the status of disease is the EDSS Expanded Disability Status Scale (EDSS).

The complete mechanism of action of IVIG is still unclear but seems to involve the modulation of expression and function Fcγ receptors, interference with complement activation, modulation of T- and B-cell activation, -differentiator and -effector functions (Ephrem et al., 2005; Kazatchkine and Kaveri, 2001; Boros et al., 2005).

While immunoglobulin prophylaxis and treatment is successfully used in many patients, among them are cases who poorly or even not respond to immunoglobulin application. Recent approaches suggest to qualitatively or quantitatively determine distinct blood cell or cell-derived factors, alone or in combination, to facilitate a individualized predictive parameter of responsiveness towards immunoglobulin therapy.

Asphalter et al in Clin Exp Immunol 2000, 121, 506-514 reported effects of in vivo IVIG replacement therapy and high-dose IVIG (2 g/kg body weight) on NK cell subsets. They describe an assay wherein intracellular IFN-γ was measured in NK cells before and after IVIG therapy (200-400 mg/kg every 3 weeks). Ruiz. et al. in Journal of Reproductive Immunology, 31 (1996), 125-141 pertains to the immunological mechanism of IVIG to inhibit NK activity in vitro when it was added to NK cytotoxicity assays using peripheral blood lymphocytes as targets. However, in both reports these findings were not related to efficacy of IVIG therapy in individual patients

Tha-In Thanyalak et al. in BLOOD, Vol 110, No 9, 9. Nov. 2007 report the effects of dendritic cells (DC's) matured for 18 hours in the presence of IVIG on natural killer (NK) cells. The effect of these cells on NK cell phenotype was examined by measuring the expression of Fc-gamma RIII after 5 days. INF-γ production and degranulation of NK cells was also analysed in co-cultures with IVIG-DC's after 48 hours and revealed increased expression of interferon-gamma. However, NK cells treated with IVIG without DC's showed only marginal activation. Thus, Tha-In concluded that only IVIG-DC's activate NK cell degranulation properly.

Kwak Joanne Y. H. et al. in EARLY PREGNANCY, Vol IV, pp 154-164 investigated the clinical effect of IVIG treatment in recurrent aborters with elevated NK cell levels while concomitantly receiving additional treatments. NK cytotoxicity and expression of CD16 was found to be significantly suppressed 5-7 days after IVIG infusion. There was no intention to use these findings to predict individual responsiveness toward IVIG therapy.

EP-A-1 801 234 relates to a diagnostic method to predict whether a subject is predisposed for acquiring a disease or developing an autoimmune disease by use of recombinant nucleic acid constructs. Such constructs are not used by the present application.

Park-Min Kyung-Hyun et al. in IMMUNITY, vol. 26, no. 1, January 2007, pp 67-78 describes several investigations related to the influence of IVIG on cellular responses to interferon-gamma. These investigations were mainly based on observations of Wisteria monocytogenes and on macrophages, which are not NK cells.

Meuer et al. describe a method to predict individual responsiveness towards immunoglobulin therapy by focusing on NK cell-associated and -derived factors like cytokines and enzymes and degranulation related parameters (WO2009/087219).

Beside several biochemical parameters, Meuer et al. reported that the amount of CD56-bright CD3-negative NK cells can decrease after immunoglobulin treatment in patients suffering from IMIDs. However, the predictive value for responsiveness towards immunoglobulin therapy has not been evaluated yet. The importance of B cells, functional immunologic and genetic factors was not addressed by this invention.

WO 2005/113815 A2 relates to the FCGR2B gene and its promoter, in particular, the invention to FCGR2B promoters with specific nucleotides at polymorphic sites. Characterization of the nucleotides at polymorphic sites is useful for characterizing the gene and the protein and is useful for determining predisposition or susceptibility to certain diseases and infections in a subject or a population of subjects. Such characterization of the gene or protein is also useful for determining immunoresponsiveness or responsiveness to therapeutic agents in a subject or population of subjects. Thus, disclosed herein are a variety of related nucleic acids, methods and tools.

WO 2011/088219 A2 discloses processes of diagnosing or treating an autoimmune abnormality whereby the presence of IgA anti-neutrophil cytoplasmic antibodies (ANCA) in a subject are detected correlating with both presence and severity of disease such as Wegener's granulomatosis (WG). The FCAR genotype predicts whether IgA ANCA will be stimulatory or inhibitory of neutrophil activation such that in subjects with an inhibitory genotype, IgA ANCA will act as an inhibitor of disease severity, and in subjects with a proinflammatory genotype, IgA ANCA will increase disease severity as observed by increased prevalence of renal disease in WG. Thus, individualized medical treatment is possible based on determination of the presence of IgA ANCA and FCAR genotype.

WO 2009/087219 A1 discloses a method of determining a patient's susceptibility for NK cell modulation by immunoglobulins in response to a treatment of a disease or prophylaxis of a disease with immunoglobulins wherein a modulation of natural killer cells caused by said immunoglobulins is determined.

The Journal of Pediatrics, Volume 147, Issue 1, Pages 78-83, July 2005 about an investigation whether reduced levels of plasma platelet-activating factor acetylhydrolase (PAF-AH) as a result of a genetic polymorphism are involved in the pathogenesis of Kawasaki disease (KD). The frequency of a V279F polymorphism (G/T transversion) in the PAF-AH gene was quantified in 76 Japanese children with KD and 112 healthy Japanese adults using the allele-specific polymerase chain reaction (PCR). Associations between genotype, clinical features, and resistance to intravenous immunoglobulin (IVIG) were investigated in the patients with KD. Plasma PAF-AH activity was measured by using [³H]-acetyl-PAF. There were no significant differences in genotype frequency between patients and controls (P=0.51). Compared with the GG (normal genotype) group, significantly more patients in the GT (heterozygous)+TT (homozygous deficient) group required additional IVIG (52% vs 14%, P=0.001). The duration of fever and maximum serum C-reactive protein (CRP) levels also were significantly increased in the GT+TT group (P=0.012 and 0.036, respectively), whereas plasma PAF-AH activity was significantly lower (P<0.0001). The authors concluded that the V279F polymorphism in the plasma PAF-AH gene and consequent enzymatic deficiency is one of the factors for IVIG nonresponse in Japanese patients with acute KD.

M. Iijima et al. report in Neurology. 2009 Oct. 27; 73(17):1348-52; about Single nucleotide polymorphism of TAG-1 influences IVIg responsiveness of Japanese patients with CIDP. Chronic inflammatory demyelinating polyneuropathy (CIDP) is characterized by immune-mediated peripheral demyelination. Although corticosteroid, IV immunoglobulin (IVIg) and plasma exchange have been established as the most effective therapeutics, subpopulations of patients show little or no response to either of these therapies. The authors examined whether particular genetic factors influence the therapeutic responsiveness of patients with CIDP. One hundred Japanese patients categorized as responders or nonresponders to IVIg therapy participated in the study. An association analysis was performed with single nucleotide polymorphisms (SNPs) and haplotype studies between the IVIg responders and nonresponders. Two separate SNPs, corresponding to TAG-1 (transient axonal glycoprotein 1) and CLEC10A (C-type lectin domain family 10, member A), showed strong significant differences between responders and non-responders. Haplotype analysis of a series of expanded SNPs, from TAG-1 or CLEC10A, showed that only TAG-1 included a significant haplotype within 1 linkage disequilibrium block, which accommodates IVIg responsiveness. Diplotype analysis of TAG-1 also supported this observation. Transient axonal glycoprotein 1 seems to be a crucial molecule involved in IV immunoglobulin responsiveness in Japanese patients with chronic inflammatory demyelinating polyneuropathy.

M. Iijima et al. report in Journal of the Peripheral Nervous System 16(Supplement):52-55 (2011) that chronic inflammatory demyelinating polyneuropathy (CIDP) is characterized by immune-mediated peripheral demyelination. Although corticosteroid, intravenous immunoglobulin (IVIg), and plasma exchange have been established as the most effective therapeutics, sub-populations of patients show little or no response to either of these therapies. The authors identified the clinical, electrophysiological, and genetic features related to IVIg responsiveness in CIDP by conducting a multi-center study. Muscle atrophy and decreased compound muscle action potential (CMAP) were pronounced in IVIg non-responders, that is, features suggesting axonal dysfunction in peripheral nerves indicated IVIg unresponsiveness in CIDP. An association analysis using single nucleotide polymorphisms (SNPs) and haplotype studies was performed between the IVIg responders and non-responders. SNPs of candidate genes were assessed that are particularly related to the function of Ranvier's node, paranode, or juxtaparanode. Two separate SNPs, corresponding to transient axonal glycoprotein-1 (TAG-1) and C-type lectin domain family 10, member A (CLEC10A), showed significant differences between responders and non-responders. Haplotype analysis of a series of expanded SNPs, from TAG-1 or CLEC10A, showed that only TAG-1 included a significant haplotype within one linkage disequilibrium block that accommodates IVIg responsiveness. Diplotype analysis of TAG-1 also supported this observation. In conclusion, SNPs in TAG-1, which is a key molecule for axon-Schwann cell interactions and is distributed at the juxtaparanode, are related to the IVIg responsiveness of CIDP patients.

C Jacobi et al. report in Clin Immunol 2009 Vol: 133:393-401 that exposure of NK cells to intravenous immunoglobulin induces IFN gamma release and degranulation but inhibits their cytotoxic activity. The mechanisms underlying the modulation of Natural Killer (NK) cell functions by intravenous immunoglobulin (IVIg) are poorly understood. Using an ex vivo whole blood assay system we demonstrate that IVIg suppresses NK cell cytotoxicity. This was paralleled by IVIg-induced degranulation of CD56(bright), CD16(positive) NK cells, reduced expression of CD16 and elevated IFN gamma release. To assess whether these findings also occur in vivo the authors analyzed whole blood before and after IVIg therapy of patients. Following IVIg treatment the number of NK cells in peripheral blood dropped significantly. Observed was reduced CD16 expression, elevated IFN gamma-amounts in plasma, reduced NK cell cytotoxicity, and granzyme B release into the plasma, confirming in vitro data. These effects on the functions of NK cells describe a novel immunomodulatory effect of IVIg. The disclosed in vitro assays could represent informative test systems to monitor effects of in vivo IVIg treatment at an individual level.

In the review article predicting treatment responses to IV immunoglobulins: Can we already ask the genes? Lehmann et al. point to the relevance of methods for finding responders and non-responders and summarize the state of the art. They refer in particular to the article of M. Iijima et al. in Neurology. 2009 Oct. 27; 73(17):1348-52.

### BRIEF DESCRIPTION OF THE INVENTION

One object of the invention is to provide a reliable method to predict individual responsiveness towards immunoglobulin therapy, in particular to therapy with immunoglobulin G.

Another object of the invention was to provide a method to predict individual responsiveness towards immunoglobulin therapy, in particular to therapy with immunoglobulin G which method is faster than known methods.

The technical problem underlying the invention is solved by a method for generating a reference for determining the likelihood of response of a patient, suffering from a disease, towards immunoglobulin therapy comprising the steps of
a) providing samples of a sufficient number of individuals, in particular at least 10 individuals, the samples containing B- and T-lymphocytes, natural killer cells, invariant T-cells and monocytes of the individuals;
b) determination of values of immune parameters which are either static, such as leukocyte subpopulations and cytokine-level in the plasma or functional, like gene expression and cytokine release after lipopolysaccharide (LPS) and/or IVIG stimulation ex vivo;
c) the determined values derived from immune parameters of the samples of the individuals are ordered in quartiles and the values belonging to the 1. quartile, values distributed at the low end of the corresponding parameter, are set to “4”, whereas values distributed in the 4. quartile are set to “+1” for an individual LDA-score calculation and values in between are set to “0”;
d) genotyping of at least two of the polynucleotides selected from the group consisting of MTM1, EIF3E, COPS8, ADAMTSL1, CXXC4, RSPO2, OR9Q1, ADAMTS9, KLHDC8A and PRDM9, in the samples of the individuals and awarding the value of 0 for specific homozygous SNP combinations (SNP—Single Nucleotide Polymorphism), which indicates that the blood sample stems from a person which will respond to immunoglobulin (IG) treatment, while awarding the value of 1 for SNP combinations not meeting that criteria, which indicates that the blood sample stems from a person which will not respond to immunoglobulin treatment;
e) combining the results of genotyping with results of immuno parameter determination in the calculation of an individual LDA-score (LDA—Linar Discriminant Analysis);
f) combining a multiplicity of individual LDA-scores to create a reference Responder Score, which allows discrimination between responders and non-responders.

The invention also concerns a method for determining the likelihood of response of a patient, suffering from a disease, towards immunoglobulin therapy comprising the steps of
a) providing a sample of the patient containing B- and T-lymphocytes, natural killer cells, invariant T-cells and monocytes;
b) determination of values of immune parameters which are either static, such as leukocyte subpopulations and cytokine-level in the plasma or functional, like gene expression and cytokine release after lipopolysaccharide (LPS) and/or IVIG stimulation ex vivo;
c) genotyping of at least two of the polynucleotides selected from the group consisting of MTM1, EIF3E, COPS8, ADAMTSL1, CXXC4, RSPO2, OR9Q1, ADAMTS9, KLHDC8A and PRDM9, and awarding the value of 0 for specific homozygous SNP combinations (SNP—Single Nucleotide Polymorphism);
d) combining the results of genotyping of step c) with results of immuno parameter determination of step b) to calculate a the patient's LDA-score (LDA—Linear Discriminant Analysis);
e) combining the patient's LDA-scores by addition to obtain a patient's Responder score;
f) comparing said patient's Responder score with the Responder score of the reference generated by the aforementioned method, wherein the cut-off to discriminate between responders and non-responders is set by adding 4 times the standard deviation of the reference data to the highest obtained score in the reference responder group;
g) and a patient's Responder score below this cut-off indicates a responder, whereas a patient's Responder score above this cut-off indicates a non-responder.

The methods of the present invention avoid the long-lasting assays as for example disclosed by Tha-In (48 hours and 5 days) performed with different cells compared to the present invention (IVIG-DC's are not used by the present invention). Furthermore Tha-In does not find an indication to use the findings for the determination of individual responsiveness towards IVIG therapy. In addition, the only comparable assay disclosed in Tha-In (NK cells treated with IVIG) showed contradictory results compared to the present invention, unfortunately, no specific conditions were published, which makes a substantial comparison impossible (page 3257, left column, lines 3-7).

The term “genotyping” is used as the skilled person understands. In particular the term means the process of determining the genotype of an individual by examining the individual's DNA sequence by using biological assays. More accurately, genotyping is the use of DNA sequences to define biological populations, by use of molecular tools current methods of genotyping include PCR, DNA sequencing, Allele specific oligonucleotide (ASO) probes, and hybridization to DNA microarrays or beads.

The terms “MTM1, EIF3E, COPS8, ADAMTSL1, CXXC4, RSPO2, OR9Q1, ADAMTS9, KLHDC8A and PRDM9” mean specific polymorphisms in the mentioned genomic segments of an individual.

Genotyping was performed with whole blood on the GeneChip® Human Mapping 6.0 Array from Affymetrix according to the protocol of the manufacturer.

“Linar Discriminant Analysis” (LDA) is a method in statistics, pattern recognition and machine learning to find a linear combination of features which characterize or separate two or more classes of objects or events. Software is commercially available under the trade name IBM® SPSS® Statistics.

In particular, according to the invention the method for generating a reference comprises the steps of
providing a sample containing B- and T-lymphocytes, natural killer cells, invariant T-cells and monocytes of the individual;
determination of immune parameters CD45, CD3, CD56, CD16, CD19, CD 14, CXCL2, CXCL8, CXCL9, CXCL10, CCL2, CCL13, ICAM1, sICAM1, CD16, IL1RA, CCL2, IL-1β, IFNγ, Actin-β, PPIB by known methods, wherein CXCL8, CXCL9, CCL2, CCL13 and ICAM1 are determined by ELISA methods and, CD16, IL-1RA, CCL2, CXCL2, CXCL8, CXCL10, IL-1β, IFNγ, Actin-β and PPIB are determined by PCR. Quartiles are calculated for all immune parameters and appropriately rounded. Values belonging to the 1. quartile, i.e. values distributed at the low end of the corresponding parameter, are set to “4”, whereas values distributed in the 4. quartile are set to “+1” in the LDA-score. Values in between are set to “0”. This simplified scoring approach implies that only individual values at the edges of their respective distribution will influence the final score. In addition, this approach allows a more robust cut-off value selection in the following linear discriminant analysis;
genotyping of at least two of the polynucleotides selected from the group consisting of MTM1, EIF3E, COPSE, ADAMTSL1, CXXC4, RSPO2, OR9Q1, ADAMTS9, KLHDC8A and PRDM9, and awarding the value of 0 for specific homozygous SNP combinations (Single Nucleotide Polymorphism), which suggest that the blood sample stems from a person which will respond to immunoglobulin (IG) treatment, while awarding the value of 1 in the LDA-score for SNP combinations not meeting that criteria, which suggests that the blood sample stems from a person which will not respond to immunoglobulin treatment;
to distinguish between potential responders and non-responders each of the genotypes are combined with the corresponding scores of the immune parameters obtained during the screening visit using linear discriminant analysis in a stepwise approach. It was decided to add a maximum of 4 additional parameters as previous studies suggested that combinations with more than 5 parameters are less robust upon (cross)validation. For the genotypes that distinguish the two groups with a p-value of ≦2*10⁻⁶ (KLHDC8A, PRDM9 and ADAMTS9) no parameters are included for an individual LDA-score, they are thus included in the final calculation unmodified;
combination of a multiplicity of individual LDA-scores, wherein all individual LDA-scores should contain values derived from a different SNP. Responders ideally score in none of the tests, i.e. their combined LDA-score is “0”, while non-responders ideally score in all individual LDA-scores with “1”, their combined LDA-score will thus represent the product of “1” by the number of individual LDA-scores applied. An ideal outcome for a non-responder will consequently be 9, when the combined LDA-score consists of 9 individual LDA-scores.

The homozygous SNP combination of interest related to the KLHDC8A gene is represented by dbSNP RS ID's rs7549293-rs10751436-rs913723-rs913722 (KLHDC8A—physical positions 205312280-205318524-205318854-205318983; equivalent to SNP_A-2182772-SNP_A-8663719-SNP_A-8663720-SNP_A8663721 (Probeset ID of Affymetrix)) with the SNP combination CC-TT-AA-TT (in the same order as given dbSNP RS ID's) indicating a non-responder and contributing “1” for individual LDA-score calculation).

The homozygous SNP combination of interest related to the PRDM9 and OR9Q1 genes is represented by dbSNP RS ID's rs13182871-rs7701403-rs12576939-rs1376486 (PRDM9—physical positions 23329734-23344192-57950654-57956832; equivalent to SNP_A-2228198-SNP_A-4217734-SNP_A-8671980-SNP_A-4215095 (Probeset ID of Affymetrix)) with the SNP combination GG-AA-TT-GG (in the same order as given dbSNP RS ID's) indicating a non-responder and contributing “1” for individual LDA-score calculation).

The homozygous SNP combination of interest related to the ADAMTS9 gene is represented by dbSNP RS ID's rs9820942-rs6780659-rs6445415-rs11721258-rs11707584-rs7652817-rs13079218-rs9819183 (ADAMTS9—physical positions 64560013-64595571-64602006-64605119-64612402-64614313-64617371-64620883; equivalent to SNP_A-4303621-SNP_A-2083737-SNP_A-8331608-SNP_A-4294272-SNP_A-1837770-SNP_A-8493544-SNP_A-8697971-SNP_A2127513 (Probeset ID of Affymetrix)) with the SNP combination GG-CC-AA-TT-AA-GG-TT-AA (in the same order as given dbSNP RS ID's) indicating a non-responder and contributing “1” for individual LDA-score calculation).

The homozygous SNP combination of interest related to the MTM1 gene is represented by dbSNP RS ID's rs222382-rs222397-rs222396-rs222385 (MTM1—physical positions 149757452-149782156-149782431-149799838; equivalent to SNP_A-8363661-SNP_A-8339704-SNP_A-8289402-SNP_A-8655121 (Probeset ID of Affymetrix)) with the SNP combination GG-AA-AA-TT (in the same order as given dbSNP RS ID's) indicating a responder and contributing “0” for individual LDA-score calculation).

The homozygous SNP combination of interest related to the EIF3E gene is represented by dbSNP RS ID's rs617285-rs596747-rs16877589 (EIF3E—physical positions 109215910-109240695-109280487; equivalent to SNP_A-1912635-SNP_A1994615-SNP_A-8652613 (Probeset ID of Affymetrix)) with the SNP combination TT-AA-TT (in the same order as given dbSNP RS ID's) indicating a responder and contributing “0” for individual LDA-score calculation).

The homozygous SNP combination of interest related to the COPS8 gene is represented by dbSNP RS ID's rs1391108-rs10194251-rs12477269-rs6732507 (COPS8—physical positions 238213597-238214023-238214279-238215383; equivalent to SNP_A-4279980-SNP_A-4245238-SNP_A-8506482-SNP_A2078620 (Probeset ID of Affymetrix)) with the SNP combination AA-TT-CC-AA (in the same order as given dbSNP RS ID's) indicating a responder and contributing “0” for individual LDA-score calculation).

The homozygous SNP combination of interest related to the ADAMTSL1 gene is represented by dbSNP RS ID's rs507741-rs550436-rs577994-rs560843-rs506193 (ADAMTSL1—physical positions 18736589-18741800-18742527-18744367-18750153; equivalent to SNP_A-1996130-SNP_A-1929603-SNP_A2080158-SNP_A-1996131-SNP_A-4247549 (Probeset ID of Affymetrix)) with the SNP combination GG-GG-TT-AA-CC (in the same order as given dbSNP RS ID's) indicating a responder and contributing “0” for individual LDA-score calculation).

The homozygous SNP combination of interest related to the CXXC4 gene is represented by dbSNP RS ID's rs3017683-rs886332-rs2214397-rs2189216 (CXXC4—physical positions 105473581-105480446-105490852-105491371; equivalent to SNP_A-2191431-SNP_A-1978333-SNP_A-2165903-SNP_A4247805 (Probeset ID of Affymetrix)) with the SNP combination TT-GG-TT-GG (in the same order as given dbSNP RS ID's) indicating a non-responder and contributing “1” for individual LDA-score calculation).

The homozygous SNP combination of interest related to the RSPO2 gene is represented by dbSNP RS ID's rs446454-rs388681-rs370662-rs399085-rs442355-rs377057-rs425932-rs564791-rs404930-rs375444-rs609666 (RSPO2—physical positions 109092699-109097676-109103640-109115072-109128653-109142307-109142353-109154549-109154584-109155015-109160212; equivalent to SNP_A-1996130-SNP_A-8413532-SNP_A-8433947-SNP_A-8408033-SNP_A-2013709-SNP_A-1816617-SNP_A-2119876-SNP_A-4197379-SNP_A2121965-SNP_A-4234104-SNP_A-1994612 (Probeset ID of Affymetrix)) with the SNP combination GG-AA-TT-CC-GG-GG-AA-GG-CC-CC-CC (in the same order as given dbSNP RS ID's) indicating a responder and contributing “0” for individual LDA-score calculation).

The polynucleotides for genotyping according to the present invention have been derived from http://www.affymetrix.com/analysis/netaffx/index.affx.

More detailed information about said SNP's are presented in table 1 and can be retrieved from the SNP-database (http://www.ncbi.nlm.nih.gov/SNP/) of the National Center for Biotechnology Information.

In a further embodiment of the invention the genotyping status is complemented with parameters by determination of at least one of the amount of cytokines released from and/or their expressed genes on cells, wherein cytokines are exemplarily selected from the group of IFN-γ, IL-1β, CXCL2, CXCL8, CXCL9, CXCL10 and CCL2.

In still a further embodiment of the invention the genotyping status is complemented with parameters by determination of the amount of at least one of the proteins CD32b, CD16, sICAM-1 and ICAM-1 (Inter Cellular Adhesion Molecule 1) released from and/or or their expressed genes on cells.

In yet a further embodiment of the invention the release of said proteins and the expression of their genes is determined after ex vivo exposure of samples with immunoglobulin, in particular IgG, IgM, IgA or a combination thereof.

In particular, the genotyping, the protein release and the gene expression is determined in whole blood, blood fractions, cell fractions or plasma.

According to the invention, in particular, a sample is incubated in presence of a stimulant in at least one assay in presence of immunoglobulins and in at least one assay in absence of immunoglobulins as control and wherein the stimulant is selected from the group consisting of lipopolysaccharides (LPS), phorbol-12-myristate-13 acetate PMA)/ionomycin, monoclonal antibodies binding to receptors on leukocytes or combinations thereof.

In particular, the amount of immunoglobulins used in assays is from about 0.01 to about 100 mg/ml in particular from about 1 to about 50 mg/ml.

Typically, the method of the invention may be performed before and/or during the treatment of a patient with immunoglobulin.

In still a further embodiment of the invention the indication of responder or non-responder is confirmed by at least one additional method.

In a further embodiment of the invention any immunoglobulin product suitable for in vivo use is concerned such as those applied intravenously, subcutaneously, intramuscularly, ocularly, intrathecially, orally, topically or inhalably.

In a further embodiment of the invention the disease is selected from the group consisting of inflammatory mediated immune diseases, autoimmune diseases, allergies, graft-versus-host reactions and prevention of transplant rejection; any kind of multiple sclerosis or any other demyelinating neurological disease; or relapsing-remitting multiple sclerosis.

In a further embodiment of the invention is used for permitting to predict the probability of a relapse of a MS patient and/or the rate of progression of the disease in terms of disability and or functioning of the patient as measured by clinical scales such as, but not limited to, the expanded disability status scale (EDSS), in particular lupus erythematosus, rheumatoid arthritis or intestinal/bowel diseases such as Crohn's disease, myositis or recurrent abortion.

Subject matter of the invention is also the use of the method of the invention for facilitating the approval or recommendation of immunoglobulins by health authorities for the treatment of any kind of multiple sclerosis or any other demyelinating disease or Lupus erythematosus, rheumatoid arthritis or intestinal/bowel diseases such as Crohn's disease, myositis or recurrent abortion.

### BRIEF DESCRIPTION OF THE FIGURES

FIG. 1-FIG. 10 show in diagrams LDA-scores.

FIG. 1 depicts an individual LDA-score based on the MTM1 SNP combination.

FIG. 2 depicts an individual LDA-score based on the EIF3E SNP combination.

FIG. 3 depicts an individual LDA-score based on the COPS8 SNP combination.

FIG. 4 depicts an individual LDA-score based on the ADAMTSL1 SNP combination.

FIG. 5 depicts an individual LDA-score based on the CXXC4 SNP combination.

FIG. 6 depicts an individual LDA-score based on the RSPO2 SNP combination.

FIG. 7 depicts an individual LDA-score based on the ADAMTS9 SNP combination.

FIG. 8 depicts an individual LDA-score based on the KLHDC8A SNP combination.

FIG. 9 depicts an individual LDA-score based on the PRDM9 SNP combination.

FIG. 10 depicts the combined LDA-score, Responder Score, used for discrimination between responder and non-responder and is based on the individual LDA-scores displayed as FIGS. 1-9.

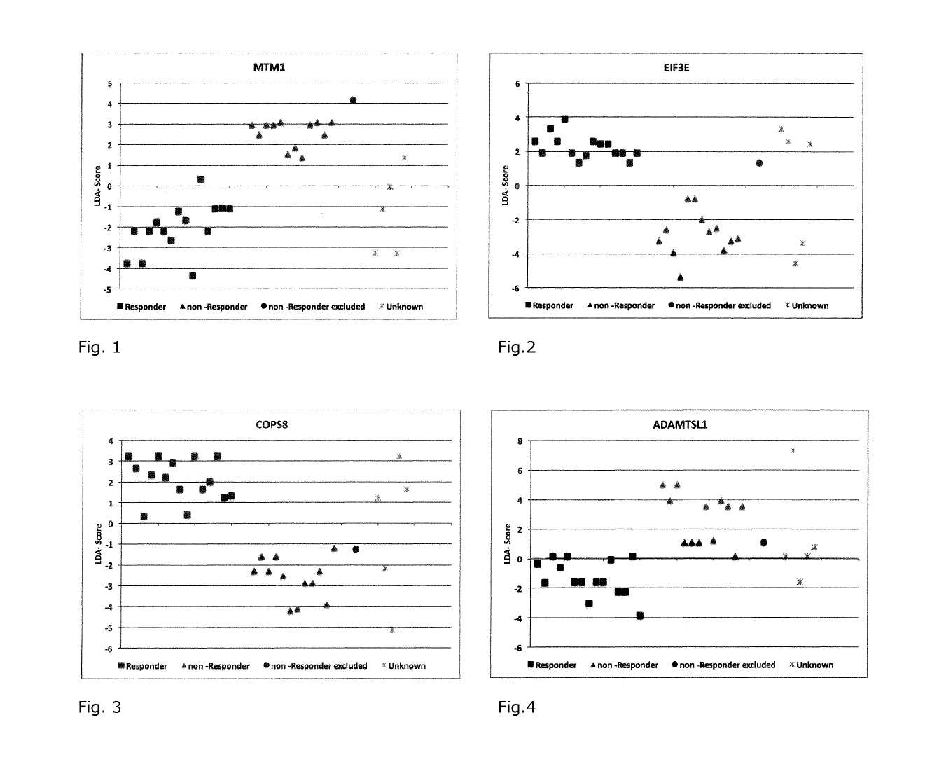

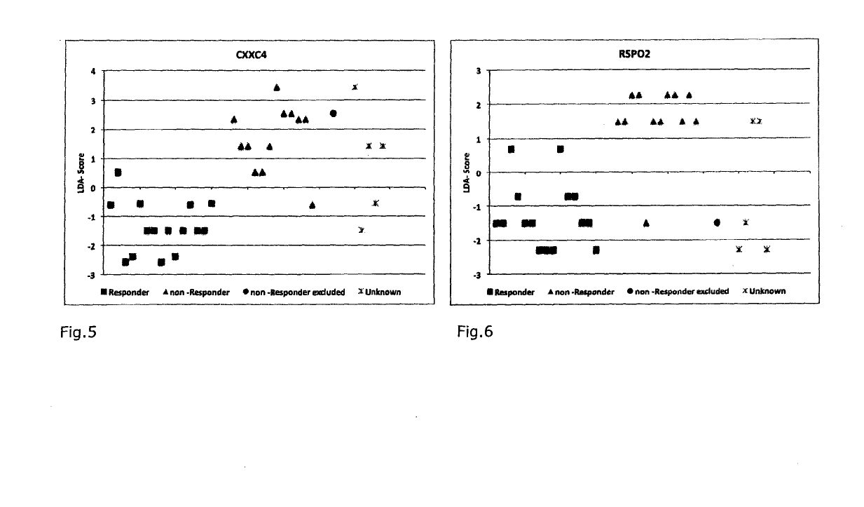

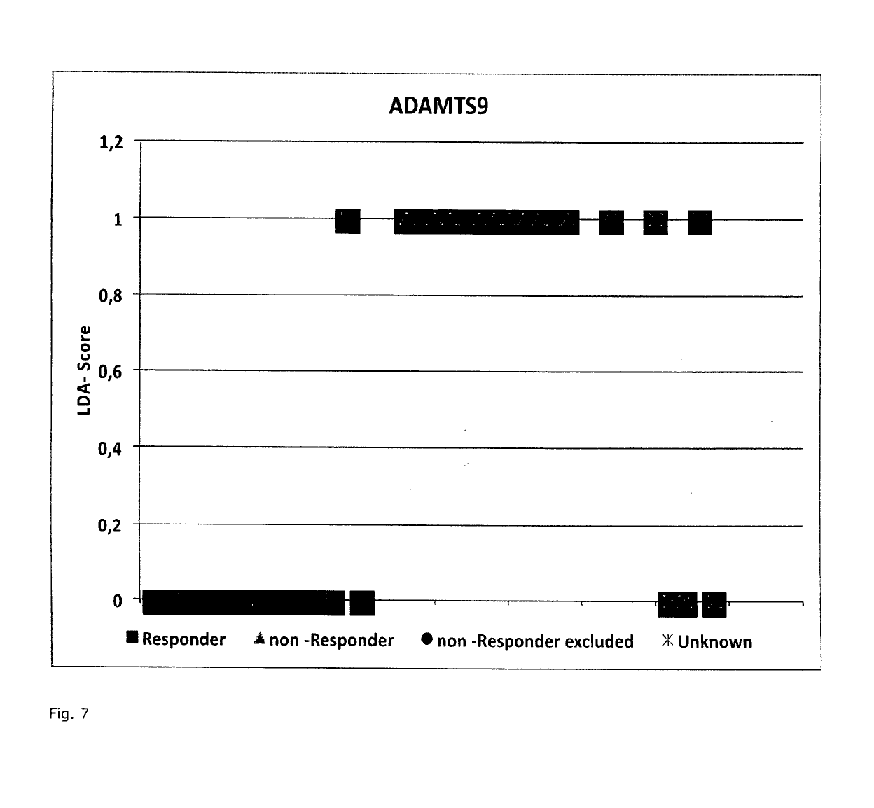

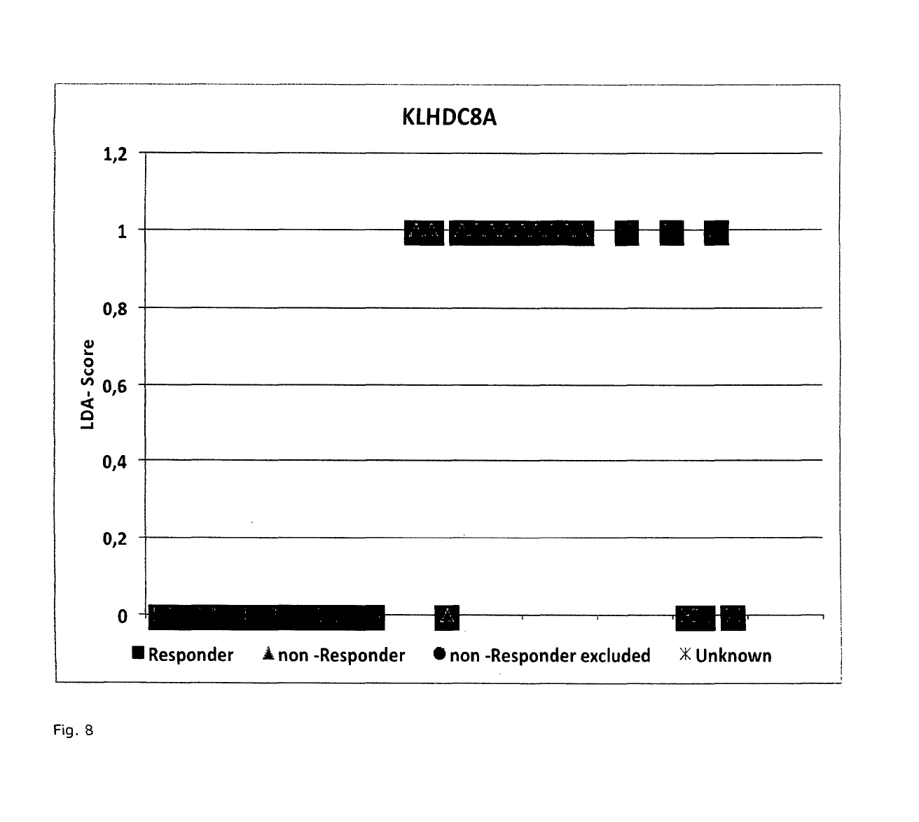

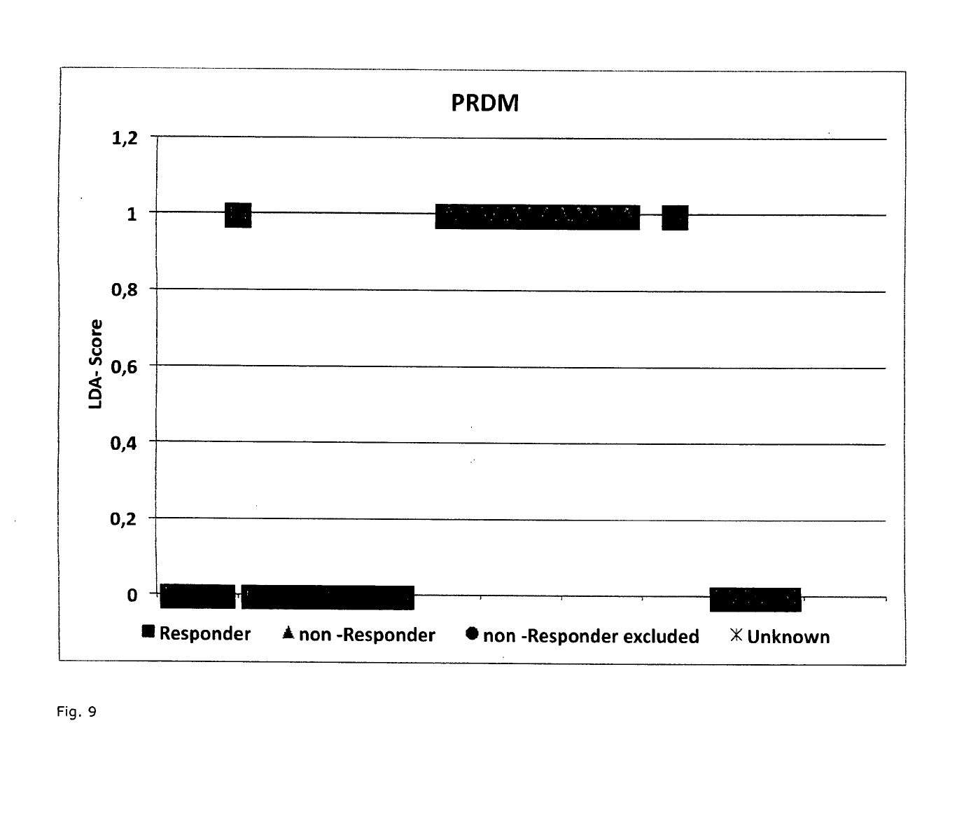

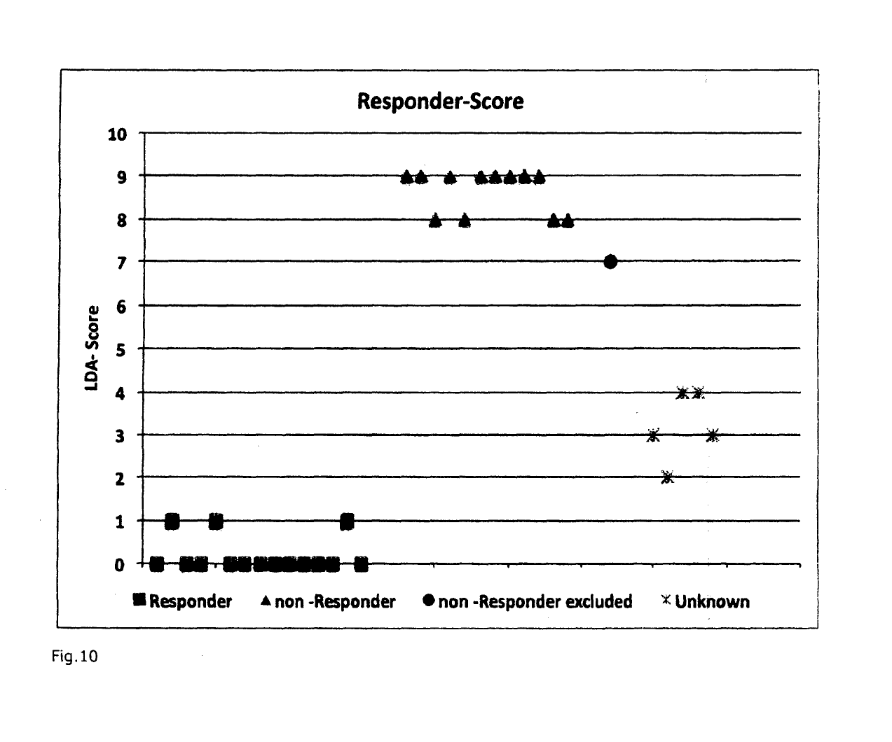

### DETAILED DESCRIPTION OF THE INVENTION

The technical problem underlying the invention is solved by a method of generating a reference for determining the likelihood of response of a patient, suffering from a disease, towards immunoglobulin therapy and a method predicting individual responsiveness towards immunoglobulin therapy, wherein the homocygocity of specific SNP's is determined and a value of “0” or “1” is awarded to predefined SNP combinations indicating responders (value “0”) and non-responders (value “1”). These values, the SNP results, are further used in calculating an individual LDA-score based on the specific SNP result and in most cases complemented by the outcome of immune parameters classified in quartiles of their analytical result. Values derived from immune parameters belonging to the 1. quartile, i.e. values distributed at the low end of the corresponding parameter, are set to “4”, whereas values distributed in the 4. quartile are set to “+1” for the individual LDA-score calculation. Values in between are set to “0”. The results of up to 4 immune parameters are combined with the result of 1 SNP combination to calculate an individual LDA-score.

These values of “4”, “0” and “+1” are multipliers for coefficients used in the calculation of an individual LDA-score. The cut-off discriminating between responders and non-responders in an individual LDA-score is determined as maximal Youden Index using Receiver Operating Characteristic analysis (ROC-Analysis). As result of an individual LDA-score a person categorised by ROC analysis as responder is awarded a “0” and a non-responder a “1” for each individual LDA-score.

As LDA (Linear Discriminant Analysis) is a statistical method it is advisable having a large data basis to obtain statistically significant values. Maximun Youden Indices and the cut-off limits given later thus indicate workable values which might change after broadening the statistical data basis. This is known to the skilled person.

A multiplicity of results of individual LDA-scores is combined, by addition, to a combined LDA-score, also called Responder Score or where appropriate patient's Responder Score, representing the final result, wherein a responder ideally scores “0”, or at least close to “0”, and a non-responder the product of “1” by the number of individual LDA-scores applied, or at least close to that number. The cut-off can be set by adding 4 times the standard deviation of the reference data to the highest obtained score in the reference responder group.

The method of the invention for generating a reference for determining the likelihood of response of a patient, suffering from a disease, towards immunoglobulin therapy comprises the steps of
a) providing samples of a sufficient number of individuals, in particular at least 10 individuals, the samples containing B- and T-lymphocytes, natural killer cells, invariant T-cells and monocytes of the individuals;
b) determination of immune parameters CD45, CD3, CD56, CD16, CD19, CD 14, CXCL2, CXCL8, CXCL9, CCL2, CCL13, ICAM1, sICAM1 (soluble ICAM1), CD16, IL-1RA, CCL2, IL-1β, IFN-γ, Actin-β, PPIB by known methods, wherein CXCL8, CXCL9, CCL2, CCL13 and ICAM1 are determined by ELISA methods and CD16, IL-1RA, CCL2, CXCL2, CXCL8, CXCL10, IL-1β, IFNγ, Actin-β and PPIB are determined by PCR. Results from gene expression were normalized by the average expression of the two housekeeping genes Actin-β and PPIB. Quartiles are calculated for all immune parameters and appropriately rounded. Values belonging to the 1. quartile, i.e. values distributed at the low end of the corresponding parameter, are set to “4”, whereas values distributed in the 4. quartile are set to “+1” in the LDA-score. Values in between are set to “0”. This simplified scoring approach implies that only individual values at the edges of their respective distribution will influence the final score;
c) the determined values derived from immune parameters of the samples of the individuals are ordered in quartiles and the values belonging to the 1. quartile, values distributed at the low end of the corresponding parameter, are set to “4”, whereas values distributed in the 4. quartile are set to “+1” for an individual LDA-score calculation and values in between are set to “0”;
d) genotyping of at least two of the polynucleotides selected from the group consisting of MTM1, EIF3E, COPS8, ADAMTSL1, CXXC4, RSPO2, OR9Q1, ADAMTS9, KLHDC8A and PRDM9, in the samples of the individuals and awarding the value of 0 for specific homozygous SNP combinations (SNP—Single Nucleotide Polymorphism), which indicates that the blood sample stems from a person which will respond to immunoglobulin (IG) treatment, while awarding the value of 1 for SNP combinations not meeting that criteria, which indicates that the blood sample stems from a person which will not respond to immunoglobulin treatment;
e) combining the results of genotyping with results of immuno parameter determination in the calculation of an individual LDA-score (LDA—Linar Discriminant Analysis);
f) combining a multiplicity of individual LDA-scores to create a reference Responder Score, which allows discrimination between responders and non-responders.

The aforementioned method of generating a reference is suitable for employing in a method for determining the likelihood of response of a patient, suffering from a disease, towards immunoglobulin therapy. Such method comprises the steps of:
a) providing a sample of the patient containing B- and T-lymphocytes, natural killer cells, invariant T-cells and monocytes;
b) determination of immune parameters CD45, CD3, CD56, CD16, CD19, CD 14, CXCL2, CXCL8, CXCL9, CCL2, CCL13, ICAM1, sICAM1 (soluble ICAM1), CD16, IL-1RA, CCL2, IL-1β, IFN-γ, Actin-β, PPIB by known methods, wherein CXCL8, CXCL9, CCL2, CCL13 and ICAM1 are determined by ELISA methods and CD16, IL-1RA, CCL2, CXCL2, CXCL8, CXCL10, IL-1β, IFNγ, Actin-β and PPIB are determined by PCR. Results from gene expression were normalized by the average expression of the two housekeeping genes Actin-β and PPIB. Quartiles are calculated for all immune parameters and appropriately rounded. Values belonging to the 1. quartile, i.e. values distributed at the low end of the corresponding parameter, are set to “4”, whereas values distributed in the 4. quartile are set to “+1” in the LDA-score. Values in between are set to “0”. This simplified scoring approach implies that only individual values at the edges of their respective distribution will influence the final score;
c) genotyping of at least two of the polynucleotides selected from the group consisting of MTM1, EIF3E, COPS8, ADAMTSL1, CXXC4, RSPO2, OR9Q1, ADAMTS9, KLHDC8A and PRDM9, and awarding the value of 0 for specific homozygous SNP combinations (SNP—Single Nucleotide Polymorphism);
d) combining the results of genotyping of step c) with results of immuno parameter determination of step b) to calculate a the patient's LDA-score (LDA—Linear Discriminant Analysis);
e) combining the patient's LDA-scores by addition to obtain a patient's Responder score;
f) comparing said patient's Responder score with the Responder score of the reference generated, wherein the cut-off to discriminate between responders and non-responders is set by adding 4 times the standard deviation of the reference data to the highest obtained score in the reference responder group;
g) and a patient's Responder score below this cut-off indicates a responder, whereas a patient's Responder score above this cut-off indicates a non-responder.
   The homozygous SNP combination of interest related to the KLHDC8A gene is represented by dbSNP RS ID's rs7549293-rs10751436-rs913723-rs913722 (KLHDC8A—physical positions 205312280-205318524-205318854-205318983; equivalent to SNP_A-2182772-SNP_A-8663719-SNP_A8663720-SNP_A-8663721 (Probeset ID of Affymetrix)) with the SNP combination CC-TT-AA-TT (in the same order as given dbSNP RS ID's) indicating a non-responder and contributing “1” for individual LDA-score calculation);
   The homozygous SNP combination of interest related to the PRDM9 and

OR9Q1 genes is represented by dbSNP RS ID's rs13182871-rs7701403-rs12576939-rs1376486 (PRDM9—physical positions 23329734-23344192-57950654-57956832; equivalent to SNP_A-2228198-SNP_A-4217734-SNP_A-8671980-SNP_A-4215095 (Probeset ID of Affymetrix)) with the SNP combination GG-AA-TT-GG (in the same order as given dbSNP RS ID's) indicating a non-responder and contributing “1” for individual LDA-score calculation);
The homozygous SNP combination of interest related to the ADAMTS9 gene is represented by dbSNP RS ID's rs9820942-rs6780659-rs6445415-rs11721258-rs11707584-rs7652817-rs13079218-rs9819183 (ADAMTS9-physical positions 64560013-64595571-64602006-64605119-64612402-64614313-64617371-64620883; equivalent to SNP_A-4303621-SNP_A2083737-SNP_A-8331608-SNP_A-4294272-SNP_A-1837770-SNP_A8493544-SNP_A-8697971-SNP_A-2127513 (Probeset ID of Affymetrix)) with the SNP combination GG-CC-AA-TT-AA-GG-TT-AA (in the same order as given dbSNP RS ID's) indicating a non-responder and contributing “1” for individual LDA-score calculation);
The homozygous SNP combination of interest related to the MTM1 gene is represented by dbSNP RS ID's rs222382-rs222397-rs222396-rs222385 (MTM1—physical positions 149757452-149782156-149782431-149799838; equivalent to SNP_A-8363661-SNP_A-8339704-SNP_A-8289402-SNP_A8655121 (Probeset ID of Affymetrix)) with the SNP combination GG-AA-AA-TT (in the same order as given dbSNP RS ID's) indicating a responder and contributing “0” for individual LDA-score calculation);
The homozygous SNP combination of interest related to the EIF3E gene is represented by dbSNP RS ID's rs617285-rs596747-rs16877589 (EIF3E-physical positions 109215910-109240695-109280487; equivalent to SNP_A1912635-SNP_A-1994615-SNP_A-8652613 (Probeset ID of Affymetrix)) with the SNP combination TT-AA-TT (in the same order as given dbSNP RS ID's) indicating a responder and contributing “0” for individual LDA-score calculation);
The homozygous SNP combination of interest related to the COPS8 gene is represented by dbSNP RS ID's rs1391108-rs10194251-rs12477269-rs6732507 (COPS8—physical positions 238213597-238214023-238214279-238215383; equivalent to SNP_A-4279980-SNP_A-4245238-SNP_A8506482-SNP_A-2078620 (Probeset ID of Affymetrix)) with the SNP combination AA-TT-CC-AA (in the same order as given dbSNP RS ID's) indicating a responder and contributing “0” for individual LDA-score calculation);
The homozygous SNP combination of interest related to the ADAMTSL1 gene is represented by dbSNP RS ID's rs507741-rs550436-rs577994-rs560843-rs506193 (ADAMTSL1—physical positions 18736589-18741800-18742527-18744367-18750153; equivalent to SNP_A-1996130-SNP_A-1929603-SNP_A-2080158-SNP_A-1996131-SNP_A-4247549 (Probeset ID of Affymetrix)) with the SNP combination GG-GG-TT-AA-CC (in the same order as given dbSNP RS ID's) indicating a responder and contributing “0” for individual LDA-score calculation);
The homozygous SNP combination of interest related to the CXXC4 gene is represented by dbSNP RS ID's rs3017683-rs886332-rs2214397-rs2189216 (CXXC4—physical positions 105473581-105480446-105490852-105491371; equivalent to SNP_A-2191431-SNP_A-1978333-SNP_A-2165903-SNP_A4247805 (Probeset ID of Affymetrix)) with the SNP combination TT-GG-TT-GG (in the same order as given dbSNP RS ID's) indicating a non-responder and contributing “1” for individual LDA-score calculation);
The homozygous SNP combination of interest related to the RSPO2 gene is represented by dbSNP RS ID's rs446454-rs388681-rs370662-rs399085-rs442355-rs377057-rs425932-rs564791-rs404930-rs375444-rs609666 (RSPO2—physical positions 109092699-109097676-109103640-109115072-109128653-109142307-109142353-109154549-109154584-109155015-109160212; equivalent to SNP_A-1996130-SNP_A-8413532-SNP_A8433947-SNP_A-8408033-SNP_A-2013709-SNP_A-1816617-SNP_A2119876-SNP_A-4197379-SNP_A-2121965-SNP_A-4234104-SNP_A1994612 (Probeset ID of Affymetrix)) with the SNP combination GG-AA-TT-CC-GG-GG-AA-GG-CC-CC-CC (in the same order as given dbSNP RS ID's) indicating a responder and contributing “0” for individual LDA-score calculation);
All SNP combinations differing from those indicated qualify for the complementing value, i.e. a “0” for responders and a “1” for non responders, to be used in the individual LDA-score calculation;
to distinguish between potential responders and non-responders each result of genotyping is combined with the corresponding scores of the immune parameters multiplied with the coefficient of said immune parameter, which was determined by linear discriminant analysis, and a constant, thereby creating an combined individual LDA-score. For the genotype combinations that distinguish the two groups with a p-value of ≦2*10⁻⁶ (KLHDC8A, PRDM9 and ADAMTS9) no parameters are included for an individual LDA-score, they are thus included in the final calculation unmodified;
combination of a multiplicity of individual combined LDA-scores, wherein all individual LDA-scores should contain values derived from a different SNP combination in order to provide a broad basis of influential factors. Responders ideally score in none of the tests, i.e. their combined LDA-score is “0”, while non-responders ideally score in all individual LDA-scores with “1”, their combined LDA-score will thus represent the product of “1” by the number of individual LDA-scores applied.

A particular embodiment of the present invention comprises the calculation of the Responder Score from 2 individual LDA-scores.

Another particular embodiment of the present invention comprises the calculation of the Responder Score from 3 individual LDA-scores.

Another particular embodiment of the present invention comprises the calculation of the Responder Score from 4 individual LDA-scores.

Another particular embodiment of the present invention comprises the calculation of the Responder Score from 5 individual LDA-scores.

Another particular embodiment of the present invention comprises the calculation of the Responder Score from 6 individual LDA-scores.

Another particular embodiment of the present invention comprises the calculation of the Responder Score from 7 individual LDA-scores.

Another particular embodiment of the present invention comprises the calculation of the Responder Score from 8 individual LDA-scores.

Another particular embodiment of the present invention comprises the calculation of the Responder Score from 9 individual LDA-scores.

A preferred outcome for a non-responder will consequently be 9, when the combined LDA-score consists of 9 individual LDA-scores. The cut-off for discrimination between responders and non-responders was determined by adding 4 times the standard deviation to the highest obtained score in the responder group of a prospective, exploratory study, which provided data used herein. The cut-off was therefore set to 4.67. In conclusion, patients are considered as potential non-responders if their combined LDA-score (patient's Responder Score) is equal to or exceeds 5 when determined by 9 individual LDA-scores.

The amount of cytokines released from or their expressed genes on cells is determined for classification into quartiles. The term cytokine is to be understood as including its subgroup chemokines. The cytokines, respectively their expressed genes, are selected from the group of Interferon-gamma (IFN-γ), C-X-C motif chemokine 2 (CXCL2), Interleukin-8 (IL-8 or CXCL8), Interleukin-1β (IL1β), C-X-C motif chemokine 9 (CXCL9), C-X-C motif chemokine 10 (CXCL10 or Interferon gamma-induced protein 10 kDa (IP-10)), chemokine C-C motif ligand 2 (CCL2) and chemokine C-C motif ligand 8 (CCL8) and are used for prediction of IgG responsiveness alone or in combination with others. Also of interest is the release and/or gen-expression of CD32b, CD16 (FcR-γ III), CD 19, CD20, CD56, IL-6R (Interleukin-6 receptor), sICAM-1 and ICAM-1 (Inter Cellular Adhesion Molecule 1).

### Combination of Parameters and Coefficients Necessary for Calculation of Individual LDA-Scores

Combination with MTM1 Genotype

Individual MTM1-LDA-score=4.664*(MTM1 genotype result)+1.553*(IG induced (netto)CD16 gene expression)−1.063*(CXCL2 plasma concentration)−2.168*(LPS induced CCL2 gene expression ratio)−2.185

Values lower than the maximal Youden Index indicate responders and are awarded a “0” for the Responder Score calculation, while values above indicate non-responders and are awarded a “1”. A workable cut-off with a maximal Youden Index was 0.843.

Combination with EIF3E Genotype

Individual EIF3E-LDA-score=−5.133*(EIF3E genotype result)+1.434*(IG induced (netto)CCL2 gene expression)+1.988*(IG/LPS induced CCL2 gene expression ratio)−0.684*(Proportion of T-cells)+1.901

Values higher than the maximal Youden Index indicate responders and are awarded a “0” for the Responder Score calculation, while values below indicate non-responders and are awarded a “1”. A workable cut-off with a maximal Youden Index was 0.288.

Combination with COPS8 Genotype

Individual COPS8-LDA-score=−6.442*(COPS8 genotype result)+1.592*(IG/LPS induced CXCL8 gene expression)+0.911*(IG/LPS induced(netto)CCL2 gene expression)+1.930*(IG/LPS induced(netto)IL-1β gene expression)+3.245

Values higher than the maximal Youden Index indicate responders and are awarded a “0” for the Responder Score calculation, while values below indicate non-responders and are awarded a “1”. A workable cut-off with a maximal Youden Index was −0.432.

Combination with ADAMTSL1 Genotype

Individual ADAMTSL1-LDA-score=3.369*(ADAMTSL1 genotype result)−1.897*(LPS induced CXCL2 gene expression)+1.212*(LPS induced CXCL10 gene expression)+1.152*(LPS stimulated IL-1β gene expression)+0.859*(IG/LPS induced(netto)CXCL2 gene expression)−1.171

Values lower than the maximal Youden Index indicate responders and are awarded a “0” for the Responder Score calculation, while values above indicate non-responders and are awarded a “1”. A workable cut-off with a maximal Youden Index was 0.6264.

Combination with CXXC4 Genotype

Individual CXXC4-LDA-score=2.925*(CXXC4 genotype result)+1.992*(IG/LPS induced(netto)IFN-γ gene expression)−1.099*(IG induced(netto)CXCL9 protein release)−1.476

Values lower than the maximal Youden Index indicate responders and are awarded a “0” for the Responder Score calculation, while values above indicate non-responders and are awarded a “1”. A workable cut-off with a maximal Youden Index was 0.007.

Combination with RSPO2 Genotype

Individual RSPO2-LDA-score=2.975*(RSPO2 genotype result)+0.7941*(sICAM1 plasma concentration)−1.491

Values lower than the maximal Youden Index indicate responders and are awarded a “0” for the Responder Score calculation, while values above indicate non-responders and are awarded a “1”. A workable cut-off with a maximal Youden Index was 1.088.

“IG induced (netto) CD16 gene expression”—Differential expression of the CD16 gene in whole blood cultures after IgG stimulation determined as adjusted CD16 transcript numbers.

“CXCL2 plasma concentration”—CXCL2 concentration in plasma—“−1” for values of the 1. quartile and “+1” for values of the 4. quartile.

“LPS induced (netto) CCL2 gene expression”—Differential expression of the CCL2 gene at the screening visit in whole blood cultures after LPS stimulation determined as adjusted CCL2 transcript numbers—“−1” for values of the 1. quartile and “+1” for values of the 4. quartile.

“LPS induced CCL2 gene expression ratio”—Ratio between LPS stimulated samples and not stimulated samples—“−1” for values of the 1. quartile and “+1” for values of the 4. quartile.

“Proportion of T-cells”—Proportion of NK-cells among lymphocytes—“−1” for values of the 1. quartile and “+1” for values of the 4. quartile.

“LPS induced CXCL8 gene expression”—Expression of the CXCL8 gene in whole blood cultures after LPS stimulation determined as adjusted CXCL8 transcript numbers—“−1” for values of the 1. quartile and “+1” for values of the 4. quartile.

“IG/LPS induced (netto) CCL2 gene expression”—Differential expression of the CCL2 gene in whole blood cultures after IgG/LPS stimulation determined as adjusted CCL2 transcript numbers—“−1” for values of the 1. quartile and “+1” for values of the 4. quartile.

“IG/LPS induced (netto) IL-1β gene expression”—Differential expression of the IL-1β gene in whole blood cultures after IgG/LPS stimulation determined as adjusted IL-1β transcript numbers—“−1” for values of the 1. quartile and “+1” for values of the 4. quartile.

“LPS induced CXCL2 gene expression”—Expression of the CXCL2 gene in whole blood cultures after LPS stimulation determined as adjusted CXCL2 transcript numbers—“−1” for values of the 1. quartile and “+1” for values of the 4. quartile.

“LPS induced CXCL10 gene expression”—Expression of the CXCL10 gene in whole blood cultures after LPS stimulation determined as adjusted CXCL10 transcript numbers—“−1” for values of the 1. quartile and “+1” for values of the 4. quartile.

“LPS stimulated IL-1β gene expression”—Expression of the IL-1β gene in whole blood cultures after LPS stimulation determined as adjusted IL-1β transcript numbers—“−1” for values of the 1. quartile and “+1” for values of the 4. quartile.

“IG/LPS induced (netto) CXCL2 gene expression”—Differential expression of the CXCL2 gene in whole blood cultures after IgG/LPS stimulation determined as adjusted CXCL2 transcript numbers—“−1” for values of the 1. quartile and “+1” for values of the 4. quartile.

“IG/LPS induced (netto) IFN-γ gene expression”—Differential expression of the IFN-γ gene in whole blood cultures after IgG/LPS stimulation determined as adjusted IFN-γ transcript numbers—“−1” for values of the 1. quartile and “+1” for values of the 4. quartile

“IG induced (netto) CXCL9 protein release”—Release of the CXCL9 protein in plasma of 24 h whole blood cultures after IgG stimulation determined as difference of CXCL9 concentration [pg/ml] between IG stimulated minus unstimulated (maltose supplemented) whole blood cultures.—“−1” for values of the 1. quartile and “+1” for values of the 4. quartile.

“sICAM1 plasma concentration”—sICAM1 concentration, determined as pg/ml, in plasma without stimulation—“−1” for values of the 1. quartile and “+1” for values of the 4. quartile.

Cited cellular-biochemical parameters were studied in whole blood and derived from plasma and or leukocytes of patients suffering from relapsing-remitting multiple sclerosis by analyzing and comparing blood samples, drawn before and after administration of immunoglobulin (regular administration of 0.4 g IVIG/kg bodyweight). The study initially incorporated 33 individuals of which 6 were later excluded due to displaying obvious inflammatory activity or dropout. Analytical results of remaining 27 patients, 15 were found to be responders and 12 non-responders according to study-design, were thus used for determination of relevant parameters.

All of these parameters (cytokine release, cytokine gen-expression), are generated in short-time ex vivo cultures of whole blood samples or plasma exposed to immunoglobulin (with or without LPS (lipopolysaccharide) stimulation).

### Reagents and Assays

Reagents, assays and assessment of results are well known to persons skilled in the art. Many assays were described in detail by Jacobi et. al. in Clinical Immunology (2009) 133, 393-401.

General preparation of whole blood cultures was performed by admixture of heparinized venous whole blood to the same volume of a stock solution containing 20 mg/ml IgG in culture medium (RPMI—1640, 10% FCS, L-glutamine, penicillin, streptomycin, 50 μM β-mercaptoethanol) with. This mixture was incubated at 37° C. for 3 h, when qRT-PCR, FACS and NK cell killing-assays were to be performed, and for 24 h, when ELISAs were to be performed. Such assays are called “IG induced” throughout this application. Some assays were performed with lipopolysaccharide (LPS, purchased from Sigma, St. Luis, USA) at 100 ng/ml final concentration in the mixture of whole blood, IgG and culture medium for stimulation. Such assays are called “IG/LPS induced” throughout this application. The same assays were also performed with maltose 10 mg/ml final concentration instead of IgG for comparison with IgG incubated assays. The difference between IG or IG/LPS incubated minus maltose or maltose/LPS (control samples as IG is missing) incubated assay results are denominated “(netto)” throughout this application. The quotient of (Analyte in stimulated sample)/(Analyte in not-stimulated sample) is denominated “ratio”.

Genotyping was performed with whole blood on the GeneChip® Human Mapping 6.0 Array from Affymetrix according to the protocol of the manufacturer. Homozygous SNP (Single Nucleotide Polymorphism) combinations at relevant positions were incorporated in data analysis.

Gene-expression assays were performed with whole blood by qRT-PCR. Cells were resuspended, after red cell lysis, in 400 μl MagNApure Lysis buffer (Roche Applied Science, Mannheim, Germany) and lysates were stored at −80° C. until analysis. An automated sample preparation system (MagNA-Pure, Roche Applied Science, Mannheim, Germany) was used for mRNA isolation according to the manufactures protocol. The elution volume was set to 50 μl. An aliquot of 8.2 μl RNA was reverse transcribed using a first strand cDNA synthesis kit (Roche Applied Science, Mannheim, Germany) and oligo- (dT) as primer using the manufacturer's protocol in a thermocycler. After termination of the cDNA synthesis, the reaction mix was diluted to a final volume of 500 μl and stored at −20° C. until polymerase chain reaction (PCR) analysis. Parameter specific primer sets optimized for the LightCycler (Roche Applied Science, Mannheim, Germany) were developed and purchased from SEARCH-LC GmbH (Heidelberg, Germany). The PCR was performed with the LightCycler FastStart DNA Sybr Green I kit (Roche Applied Science, Mannheim, Germany) according to the protocol provided in the parameter specific kits. RNA input was normalized by the average expression of the two housekeeping genes β-actin and Cyclophilin B (peptidylpropyl isomerase B; PPIB). The data was used as adjusted transcripts per μl cDNA.

Analysis of released proteins was performed by ELISA in supernatants of whole blood cultures by using commercially available kits (Diaclone, Pelikine and Luminex) according to the manufacturer's protocols. Whole blood cultures were sedimented by centrifugation after 24 h of incubation at 37° C. and supernatants were kept at −80° C. until assay. The data was used as pg/ml protein concentration.

Any other change of cellular marker associated with degranulation can be utilized to detect and quantify the degranulation efficacy and status induced by IgG exposure. Representatives of such NK cell granulae (lytic lysosomes) compounds are the proteins perforin and granzymes (the latter proteases, more specifically granzyme B), which can be quantified by for instance antigen detection systems like ELISA or direct enzymatic tests (for enzymes and proteases). The increased expression of CD107a is a typical indicator of NK degranulation. Using lipopolysaccharide (bacteria derived) in a whole blood assay system and thereby mimicking a patho-physiological situation, both IFN-gamma and CXCL10 are upregulated on mRNA transcript (number) and protein (release) level. It was found that in the presence of added immunoglobulin (upon stimulation with LPS), the increase of CXCL10 was reduced by immunoglobulins as compared to the control without immunoglobulins, whereas no relative reduction was observed for IFN-γ.

In general, the monitored signal is measured by methods known to the expert, like specific detection by using a labeled antibody, fragment or affinity ligand in flow cytometry (like FACS, fluorescent-activated cell sorting).

All those analytical results were correlated with the outcome of the study, differentiation between patients responding to IgG-treatment (“responders”) and non responding patients (“non-responders”), and subjected to Linear Discriminant Analysis (LDA) for determination of most relevant parameters for prediction of a person's susceptibility to IgG treatment. After identification of useful parameters several LDA-scores with high predictive value were established, as displayed by the following examples. Analytical results were introduced in respective formula for LDA-Sore calculation in values and dimensions as indicated in the general assay description.

### Example 1

### Calculation Involving 9 Individual LDA-Scores

Determination of SNP results
The homozygous SNP combination of interest related to the KLHDC8A gene is represented by dbSNP RS ID's rs7549293-rs10751436-rs913723-rs913722 with the SNP combination CC-TT-AA-TT (in the same order as given dbSNP RS ID's) indicating a non-responder and contributing “1” for individual LDA-score calculation);
The homozygous SNP combination of interest related to the PRDM9 and OR9Q1 genes is represented by dbSNP RS ID's rs13182871-rs7701403-rs12576939-rs1376486 with the SNP combination GG-AA-TT-GG (in the same order as given dbSNP RS ID's) indicating a non-responder and contributing “1” for individual LDA-score calculation);
The homozygous SNP combination of interest related to the ADAMTS9 gene is represented by dbSNP RS ID's rs9820942-rs6780659-rs6445415-rs11721258-rs11707584-rs7652817-rs13079218-rs9819183 with the SNP combination GG-CC-AA-TT-AA-GG-TT-AA (in the same order as given dbSNP RS ID's) indicating a non-responder and contributing “1” for individual LDA-score calculation);
The homozygous SNP combination of interest related to the MTM1 gene is represented by dbSNP RS ID's rs222382-rs222397-rs222396-rs222385 with the SNP combination GG-AA-AA-TT (in the same order as given dbSNP RS ID's) indicating a responder and contributing “0” for individual LDA-score calculation);
The homozygous SNP combination of interest related to the EIF3E gene is represented by dbSNP RS ID's rs617285-rs596747-rs16877589 with the SNP combination TT-AA-TT (in the same order as given dbSNP RS ID's) indicating a responder and contributing “0” for individual LDA-score calculation);
The homozygous SNP combination of interest related to the COPS8 gene is represented by dbSNP RS ID's rs1391108-rs10194251-rs12477269-rs6732507 with the SNP combination AA-TT-CC-AA (in the same order as given dbSNP RS ID's) indicating a responder and contributing “0” for individual LDA-score calculation);
The homozygous SNP combination of interest related to the ADAMTSL1 gene is represented by dbSNP RS ID's rs507741-rs550436-rs577994-rs560843-rs506193 with the SNP combination GG-GG-TT-AA-CC (in the same order as given dbSNP RS ID's) indicating a responder and contributing “0” for individual LDA-score calculation);
The homozygous SNP combination of interest related to the CXXC4 gene is represented by dbSNP RS ID's rs3017683-rs886332-rs2214397-rs2189216 with the SNP combination TT-GG-TT-GG (in the same order as given dbSNP RS ID's) indicating a non-responder and contributing “1” for individual LDA-score calculation);
The homozygous SNP combination of interest related to the RSPO2 gene is represented by dbSNP RS ID's rs446454-rs388681-rs370662-rs399085-rs442355-rs377057-rs425932-rs564791-rs404930-rs375444-rs609666 with the SNP combination GG-AA-TT-CC-GG-GG-AA-GG-CC-CC-CC (in the same order as given dbSNP RS ID's) indicating a responder and contributing “0” for individual LDA-score calculation);
Determination of immune parameters CD45, CD3, CD56, CD16, CD19, CD 14, CXCL8, CXCL9, CCL2, CCL13, ICAM1, CD16, IL-1RA, CCL2, IL-1β, IFNγ, Actin-β, PPIB by known methods, wherein CXCL8, CXCL9, CCL2, CCL13 and ICAM1 are determined by ELISA methods and, CD16, IL-1RA, CCL2, IL-1β, IFNγ, Actin-β and PPIB were determined by PCR. Quartiles were calculated for all immune parameters and appropriately rounded. Values belonging to the 1. quartile, i.e. values distributed at the low end of the corresponding parameter, were set to “4”, whereas values distributed in the 4. quartile were set to “+1” in the individual LDA-score. Values in between were set to “0” and up to 4 parameters were combined with the outcome of 1 SNP combination to individual LDA-scores. The SNP results of the KLHDC8A, PRDM9 and ADAMTS9 SNP combinations remained unmodified and represented their own individual LDA-score. Thus 9 individual LDA-scores were provided;
Calculation of individual LDA-scores
Combination with MTM1 genotype

Individual MTM1-LDA-score=4.664*(MTM1 genotype result)+1.553*(IG induced(netto)CD16 gene expression)−1.063*(CXCL2 plasma concentration)−2.168*(LPS induced CCL2 gene expression ratio)−2.185
Combination with EIF3E genotype

Individual EIF3E-LDA-score=−5.133*(EIF3E genotype result)+1.434*(IG induced(netto)CCL2 gene expression)+1.988*(IG/LPS induced CCL2 gene expression ratio)−0.684*(Proportion of T-cells)+1.901
Combination with COPS8 genotype

Individual COPS8-LDA-score=−6.442*(COPS8 genotype result)+1.592*(IG/LPS induced CXCL8 gene expression)+0.911*(IG/LPS induced (netto)CCL2 gene expression)+1.930*(IG/LPS induced(netto)IL-1β gene expression)+3.245
Combination with ADAMTSL1 genotype

Individual ADAMTSL1-LDA-score=3.369*(ADAMTSL1 genotype result)−1.897*(LPS induced CXCL2 gene expression)+1.212*(LPS induced CXCL10 gene expression)+1.152*(LPS stimulated IL-1β gene expression)+0.859*(IG/LPS induced(netto)CXCL2 gene expression)−1.171
Combination with CXXC4 genotype

Individual CXXC4-LDA-score=2.925*(CXXC4 genotype result)+1.992*(IG/LPS induced(netto)IFN-γ gene expression)−1.099*(IG induced (netto)CXCL9 protein release)−1.476
Combination with RSPO2 genotype

Individual RSPO2-LDA-score=2.975*(RSPO2 genotype result)+0.7941*(sICAM1 plasma concentration)−1.491
The Responder Score responsible for the discrimination between responder and non-responder was established by combining the results of the 9 individual LDA-scores, i.e. the 6 individual LDA-scores cited just prior and the 3 unmodified SNP results of the KLHDC8A, PRDM9 and ADAMTS9 genotypes. Application of the cut-off limit indicated patients with a score of at least 5 as potential non-responders, while those scoring less than 5 are considered as potential responders.

### Example 2

Calculations involving 2-8 LDA-scores are performed likewise to example 1.

This method allows the identification of persons responding/non-responding to any immunoglobulin product suitable for in vivo use such as those applied intravenously, subcutaneously, intramuscularly, ocularly, intrathecially, orally, topically or inhalably for diseases which are in principle accessible to immunoglobulin treatment, such as immune mediated inflammatory diseases, autoimmune diseases, allergies, graft-versus-host reactions and prevention of transplant rejection; any kind of multiple sclerosis or any other demyelinating neurological disease; or relapsing-remitting multiple sclerosis.

The method also permits to predict the probability of a relapse of a MS patient and/or the rate of progression of the disease in terms of disability and or functioning of the patient as measured by clinical scales such as, but not limited to, the expanded disability status scale (EDSS), in particular lupus erythematosus, rheumatoid arthritis or intestinal/bowel diseases such as Crohn's disease, myositis or recurrent abortion.

This method can additionally be used for facilitating the approval or recommendation of immunoglobulins by health authorities for the treatment of any kind of multiple sclerosis or any other demyelinating disease or Lupus erythematosus, rheumatoid arthritis or intestinal/bowel diseases such as Crohn's disease, myositis or recurrent abortion.

TABLE 1

| Gene | Physical | dbSNP | | SEQ ID | Strand vs. |
|---|---|---|---|---|---|
| Symbol | Position | RS ID | Flank | No | dbSNP |
| KLHDC8A | 205312280 | rs7549293 | CAGATGGAAACCAACA[C/G]GCATTTGCTGTGGGCT | 1 | reverse |
| PRDM9 | 23329734 | rs13182871 | CTACTCAGCAGAATAC[A/G]AGCAGAGGTCAGAGAA | 2 | reverse |
| OR9Q1 | 57950654 | rs12576939 | CTTTATTGAGGTATAA[A/T]TGCTATGCAAAAAAAA | 3 | reverse |
| ADAMTS9 | 64560013 | rs9820942 | GCTCAGGAGCTATTAG[A/G]ATTTTTTTTGTATCGA | 4 | same |
| ADAMTS9 | 64620883 | rs9819183 | ATAACAGTATCAGGAC[A/G]AGCTTCTCTAGGAGGG | 5 | reverse |
| MTM1 | 149757452 | rs222382 | AACAAATACCTGTTAA[A/G]TATCTTTTGGGTGCCA | 6 | reverse |
| EIF3E | 109215910 | rs617285 | TGTGCCTATTGACTTG[C/T]ACTATGCACGACCTAT | 7 | reverse |
| COPS8 | 238213597 | rs1391108 | AGAGGCAGAATCACCA[A/G]TGACTCATACCCCTTG | 8 | same |
| ADAMTSL1 | 18736589 | rs507741 | CAGAGCTACCCTGCTG[A/G]AGGAGTTAGGTATGCA | 9 | reverse |
| CXXC4 | 105473581 | rs3017683 | CATATCTATGCTTCCA[C/T]GCTCATGGTATATAAG | 10 | reverse |
| RSPO2 | 109092699 | rs446454 | CCATCCACATCCCAAA[G/T]CACTGACAGTCTAACT | 11 | reverse |
| RSPO2 | 109142353 | rs425932 | AGTCAACCTAAAATAA[A/G]ATTGGCAACAGTTAAA | 12 | same |
| KLHDC8A | 205318524 | rs10751436 | AGTGGGTATCCATTAA[C/T]GAATGCATCTGTTCAT | 13 | same |
| KLHDC8A | 205318854 | rs913723 | TCATGCTGGTGTTTCA[A/G]GTTTCTGACATTGCTG | 14 | same |
| KLHDC8A | 205318983 | rs913722 | ACGATGAAGGCTATGA[C/T]CTCCAGCCTACGTTTT | 15 | reverse |
| CDH12 | 23344192 | rs7701403 | aacatcttttaatatc[A/C]ctgttggtcacttgta | 16 | rev/T |
| OR9Q1 | 57956832 | rs1376486 | ggacacctggcttcta[A/G]ttcacccacactgcat | 17 | fwd/T |
| ADAMTS9 | 64595571 | rs6780659 | ACTACAGTGATCAAAA[C/T]AGTCTACTGGCATAAG | 18 | same |
| ADAMTS9 | 64602006 | rs6445415 | TAAAATCTAATTTTAA[A/G]CCAACATATCCATGTG | 19 | same |
| ADAMTS9 | 64605119 | rs11721258 | CGGAAATCTAAATCTG[C/T]ACCTAGATCCTGTGCT | 20 | reverse |
| ADAMTS9 | 64612402 | rs11707584 | TACTTTTATAAGCCCA[A/G]GAAAGATCCCAACCCA | 21 | same |
| ADAMTS9 | 64614313 | rs7652817 | TTAATTGTCTTATAAA[A/G]TTGTCACTCATTTGGA | 22 | same |
| ADAMTS9 | 64617371 | rs13079218 | GATGGATAATCCACCA[C/T]AACGAGACTGATTTTT | 23 | same |
| MTM1 | 149782156 | rs222397 | tcacctatatcctgga[A/G]cggttgtgaggataaa | 24 | rev/T |
| MTM1 | 149782431 | rs222396 | cccccactagagacca[A/C]gcatctggaaatggtg | 25 | fwd/T |
| MTM1 | 149799838 | rs222385 | ctgagataattcctta[G/T]aagtctgagtagggaa | 26 | fwd/B |
| EIF3E | 109240695 | rs596747 | ttaattacagttttga[A/T]gttagtgatgtttgtc | 27 | rev/ |
| EIF3E | 109280487 | rs16877589 | ctccttctgacactgg[C/T]aaaactgtgaaggtgt | 28 | rev/B |
| COPS8 | 238214023 | rs10194251 | gtgggtctaaacacaa[C/T]gagatggaaggagaca | 29 | rev/B |
| COPS8 | 238214279 | rs12477269 | ttagcatgtaccctta[C/T]atgtcattggccaagt | 30 | rev/B |
| COPS8 | 238215383 | rs6732507 | accatgtgacaactga[A/C]agatgccatgttaagc | 31 | rev/T |
| ADAMTSL1 | 18741800 | rs550436 | ttcatagattcacaga[A/G]gggaagatagccatgc | 32 | rev/T |
| ADAMTSL1 | 18742527 | rs577994 | accgtccttgagattc[C/T]attggatttacttcca | 33 | rev/B |
| ADAMTSL1 | 18744367 | rs560843 | aatgcttagcaatgaa[A/T]gcttaaatgagaaact | 34 | fwd/ |
| ADAMTSL1 | 18750153 | rs506193 | tacaggtattatgctt[A/C]aaaaaaggttttcttt | 35 | rev/T |
| CXXC4 | 105480446 | rs886332 | tgaactggaccttgaa[A/G]gataatcattttctag | 36 | fwd/T |
| CXXC4 | 105490852 | rs2214397 | caaagtgtatactcaa[G/T]gactaattaggatgct | 37 | rev/B |
| CXXC4 | 105491371 | rs2189216 | tactacccccagatta[A/G]cagcctagcaggtact | 38 | rev/T |
| RSPO2 | 109097676 | rs388681 | tctgtaaatttaacta[A/T]ggaaggcagtggctaa | 39 | rev/ |
| RSPO2 | 109103640 | rs370662 | tgggaacaggaactaa[C/T]tggacctttggggatg | 40 | fwd/B |
| RSPO2 | 109115072 | rs399085 | ggccaagacataccaa[C/T]gaggcaggaaactatg | 41 | rev/B |
| RSPO2 | 109128653 | rs442355 | gcttttttccagatcc[C/G]aaaacccttccattca | 42 | fwd/ |
| RSPO2 | 109142307 | rs377057 | gttataaggggacaaa[A/G]gacggtagcttgtatt | 43 | rev/T |
| RSPO2 | 109154549 | rs564791 | tagatcacctttgggg[A/G]aatcttggcactaatg | 44 | fwd/T |
| RSPO2 | 109154584 | rs404930 | agtgagtcttgacaac[C/T]aaaatggtgtgaagtg | 45 | fwd/B |
| RSPO2 | 109155015 | rs375444 | ctaaggcaatgaggac[A/C]ctagagtctgcttttg | 46 | rev/T |
| RSPO2 | 109160212 | rs609666 | catgccaacatcagca[C/G]caaagatacaaaggta | 47 | rev/ |

| Gene | Physical | dbSNP | Allele | SNP | | Gene | |
|---|---|---|---|---|---|---|---|
| Symbol | Position | RS ID | A/B | Chromosome | Cytoband | Relationship | Probe Set ID |
| KLHDC8A | 205312280 | rs7549293 | C/G | 1 | q32.1 | intron | SNP_A-2182772 |
| PRDM9 | 23329734 | rs13182871 | A/G | 5 | | | SNP_A-2228198 |
| OR9Q1 | 57950654 | rs12576939 | A/T | 11 | | | SNP_A-8671980 |
| ADAMTS9 | 64560013 | rs9820942 | A/G | 3 | p14.1 | intron | SNP_A-4303621 |
| ADAMTS9 | 64620883 | rs9819183 | A/G | 3 | p14.1 | intron | SNP_A-2127513 |
| MTM1 | 149757452 | rs222382 | A/G | X | | | SNP_A-8363661 |
| EIF3E | 109215910 | rs617285 | C/T | 8 | | | SNP_A-1912635 |
| COPS8 | 238213597 | rs1391108 | A/G | 2 | | | SNP_A-4279980 |
| ADAMTSL1 | 18736589 | rs507741 | A/G | 9 | | | SNP_A-1996130 |
| CXXC4 | 105473581 | rs3017683 | C/T | 4 | | | SNP_A-2191431 |
| RSPO2 | 109092699 | rs446454 | G/T | 8 | | | SNP_A-2013708 |
| RSPO2 | 109142353 | rs425932 | A/G | 8 | | | SNP_A-2119876 |
| KLHDC8A | 205318524 | rs10751436 | C/T | 1 | q32.1 | intron | SNP_A-8663719 |
| KLHDC8A | 205318854 | rs913723 | A/G | 1 | q32.1 | intron | SNP_A-8663720 |
| KLHDC8A | 205318983 | rs913722 | C/T | 1 | q32.1 | intron | SNP_A-8663721 |
| CDH12 | 23344192 | rs7701403 | A/C | 5 | | | SNP_A-4217734 |
| OR9Q1 | 57956832 | rs1376486 | A/G | 11 | | | SNP_A-4215095 |
| ADAMTS9 | 64595571 | rs6780659 | C/T | 3 | p14.1 | intron | SNP_A-2083737 |
| ADAMTS9 | 64602006 | rs6445415 | A/G | 3 | p14.1 | intron | SNP_A-8331608 |
| ADAMTS9 | 64605119 | rs11721258 | C/T | 3 | p14.1 | intron | SNP_A-4294272 |
| ADAMTS9 | 64612402 | rs11707584 | A/G | 3 | p14.1 | intron | SNP_A-1837770 |
| ADAMTS9 | 64614313 | rs7652817 | A/G | 3 | p14.1 | intron | SNP_A-8493544 |
| ADAMTS9 | 64617371 | rs13079218 | C/T | 3 | p14.1 | intron | SNP_A-8697971 |
| MTM1 | 149782156 | rs222397 | A/G | X | | | SNP_A-8339704 |
| MTM1 | 149782431 | rs222396 | A/C | X | | | SNP_A-8289402 |
| MTM1 | 149799838 | rs222385 | G/T | X | | | SNP_A-8655121 |
| EIF3E | 109240695 | rs596747 | A/T | 8 | | | SNP_A-1994615 |
| EIF3E | 109280487 | rs16877589 | C/T | 8 | | | SNP_A-8652613 |
| COPS8 | 238214023 | rs10194251 | C/T | 2 | | | SNP_A-4245238 |
| COPS8 | 238214279 | rs12477269 | C/T | 2 | | | SNP_A-8506482 |
| COPS8 | 238215383 | rs6732507 | A/C | 2 | | | SNP_A-2078620 |
| ADAMTSL1 | 18741800 | rs550436 | A/G | 9 | | | SNP_A-1929603 |
| ADAMTSL1 | 18742527 | rs577994 | C/T | 9 | | | SNP_A-2080158 |
| ADAMTSL1 | 18744367 | rs560843 | A/T | 9 | | | SNP_A-1996131 |
| ADAMTSL1 | 18750153 | rs506193 | A/C | 9 | | | SNP_A-4247549 |
| CXXC4 | 105480446 | rs886332 | A/G | 4 | | | SNP_A-1978333 |
| CXXC4 | 105490852 | rs2214397 | G/T | 4 | | | SNP_A-2165903 |
| CXXC4 | 105491371 | rs2189216 | A/G | 4 | | | SNP_A-4247805 |
| RSPO2 | 109097676 | rs388681 | A/T | 8 | | | SNP_A-8413532 |
| RSPO2 | 109103640 | rs370662 | C/T | 8 | | | SNP_A-8433947 |
| RSPO2 | 109115072 | rs399085 | C/T | 8 | | | SNP_A-8408033 |
| RSPO2 | 109128653 | rs442355 | C/G | 8 | | | SNP_A-2013709 |
| RSPO2 | 109142307 | rs377057 | A/G | 8 | | | SNP_A-1816617 |
| RSPO2 | 109154549 | rs564791 | A/G | 8 | | | SNP_A-4197379 |
| RSPO2 | 109154584 | rs404930 | C/T | 8 | | | SNP_A-2121965 |
| RSPO2 | 109155015 | rs375444 | A/C | 8 | | | SNP_A-4234104 |
| RSPO2 | 109160212 | rs609666 | C/G | 8 | | | SNP_A-1994612 |

1. A method for generating a reference for determining the likelihood of response of a patient, suffering from a disease, towards immunoglobulin therapy comprising the steps of
a) providing samples of a sufficient number of individuals, the samples containing B- and T-lymphocytes, natural killer cells, invariant T-cells or monocytes of the individuals;
b) determination of values of immune parameters which are either static or functional
c) the determined values derived from immune parameters of the samples of the individuals are ordered in quartiles and the values belonging to the 1. quartile, values distributed at the low end of the corresponding parameter, are set to “4”, whereas values distributed in the 4. quartile are set to “+1” for an individual LDA-score calculation and values in between are set to “0”;
d) genotyping of at least two polynucleotides selected from the group consisting of MTM1, EIF3E, COPS8, ADAMTSL1, CXXC4, RSPO2, OR9Q1, ADAMTS9, KLHDC8A and PRDM9, in the samples of the individuals and awarding the value of 0 for specific homozygous SNP combinations (SNP—Single Nucleotide Polymorphism), which indicates that the blood sample stems from a person which will respond to immunoglobulin (IG) treatment, while awarding the value of 1 for SNP combinations not meeting that criteria, which indicates that the blood sample stems from a person which will not respond to immunoglobulin treatment;
e) combining the results of genotyping with results of immuno parameter determination in the calculation of an individual LDA-score (LDA—Linar Discriminant Analysis);
f) combining a multiplicity of individual LDA-scores to create a reference Responder Score, which allows discrimination between responders and non-responders.

2. The method of claim 1 wherein the immune parameters are selected from the group consisting of CD45, CD3, CD56, CD16, CD19, CD 14, CXCL2, CXCL8, CXCL9, CXCL10, CCL2, CCL13, ICAM1, sICAM1 (soluble ICAM1), IL-1RA, CCL2, IFN-γ, Actin-β, and PPIB.

3. The method of claim 1 wherein the homozygous SNP combination related to the KLHDC8A gene is represented by dbSNP RS ID's rs7549293-rs10751436-rs913723-rs913722 with the SNP combination CC-TT-AA-TT (in the same order as given dbSNP RS ID's) indicating a non-responder and contributing “1” for individual LDA-score calculation, while all different SNP combinations indicate a responder and contribute “0” for the individual LDA-score calculation.

4. The method of claim 1 wherein the homozygous SNP combination related to the PRDM9 and OR9Q1 genes is represented by dbSNP RS ID's rs13182871-rs7701403-rs12576939-rs1376486 with the SNP combination GG-AA-TT-GG (in the same order as given dbSNP RS ID's) indicating a non-responder and contributing “1” for individual LDA-score calculation, while all different SNP combinations indicate a responder and contribute “0” for the individual LDA-score calculation.

5. The method of claim 1 wherein the homozygous SNP combination related to the ADAMTS9 gene is represented by dbSNP RS ID's rs9820942-rs6780659-rs6445415-rs11721258-rs11707584-rs7652817-rs13079218-rs9819183 with the SNP combination GG CC AA TT AA GG TT AA (in the same order as given dbSNP RS ID's) indicating a non-responder and contributing “1” for individual LDA-score calculation, while all different SNP combinations indicate a responder and contribute “0” for the individual LDA-score calculation.

6. The method of claim 1 wherein the homozygous SNP combination related to the MTM1 gene is represented by dbSNP RS ID's rs222382-rs222397-rs222396-rs222385 with the SNP combination GG-AA-AA-TT (in the same order as given dbSNP RS ID's) indicating a responder and contributing “0” for individual LDA-score calculation, while all different SNP combinations indicate a non-responder and contribute “1” for the individual LDA-score calculation.

7. The method of claim 1 wherein the homozygous SNP combination related to the EIF3E gene is represented by dbSNP RS ID's rs617285-rs596747-rs16877589 with the SNP combination TT-AA-TT (in the same order as given dbSNP RS ID's) indicating a responder and contributing “0” for individual LDA-score calculation, while all different SNP combinations indicate a non-responder and contribute “1” for the individual LDA-score calculation.

8. The method of claim 1 wherein the homozygous SNP combination related to the COPS8 gene is represented by dbSNP RS ID's rs1391108-rs10194251-rs12477269-rs6732507 with the SNP combination AA-TT-CC-AA (in the same order as given dbSNP RS ID's) indicating a responder and contributing “0” for individual LDA-score calculation, while all different SNP combinations indicate a non-responder and contribute “1” for the individual LDA-score calculation.

9. The method of claim 1 wherein the homozygous SNP combination related to the ADAMTSL1 gene is represented by dbSNP RS ID's rs507741-rs550436-rs577994-rs560843-rs506193 with the SNP combination GG-GG-TT-AA-CC (in the same order as given dbSNP RS ID's) indicating a responder and contributing “0” for individual LDA-score calculation, while all different SNP combinations indicate a non-responder and contribute “1” for the individual LDA-score calculation.

10. The method of claim 1 wherein the homozygous SNP combination related to the CXXC4 gene is represented by dbSNP RS ID's rs3017683-rs886332-rs2214397-rs2189216 with the SNP combination TT-GG-TT-GG (in the same order as given dbSNP RS ID's) indicating a non-responder and contributing “1” for individual LDA-score calculation, while all different SNP combinations indicate a responder and contribute “0” for the individual LDA-score calculation.

11. The method of claim 1 wherein the homozygous SNP combination related to the RSPO2 gene is represented by dbSNP RS ID's rs446454-rs388681-rs370662-rs399085-rs442355-rs377057-rs425932-rs564791-rs404930-rs375444-rs609666 with the SNP combination GG AA TT CC GG GG AA GG CC-CC-CC (in the same order as given dbSNP RS ID's) indicating a responder and contributing “0” for individual LDA-score calculation, while all different SNP combinations indicate a non-responder and contribute “1” for the individual LDA-score calculation.

12. The method of claim 1 wherein the multiplicity of immune parameters used for the creation of individual LDA-scores is 4 at maximum.

13. The method of claim 1 wherein the genotyping result of the polynucleotides related to the genes of ADAMTS9, KLHDC8A and PRDM9 are not complemented for creation of individual LDA-scores and represent their own individual LDA-score.

14. The method of claim 1 wherein the individual LDA-score related to the MTM1 genotype is calculated by the formula:
Individual MTM1-LDA-score=4.664*(MTM1 genotype result)+1.553*(IG induced(netto)CD16 gene expression)−1.063*(CXCL2 plasma concentration)−2.168*(LPS induced CCL2 gene expression ratio)−2.185,
wherein values lower than the maximal Youden Index indicate responders and are awarded a “0” for the Responder Score calculation, while values above indicate non-responders and are awarded a “1”.

15. The method of claim 1 wherein the individual LDA-score related to the EIF3E genotype is calculated by the formula:
Individual EIF3E-LDA-score=−5.133*(EIF3E genotype result)+1.434*(IG induced(netto)CCL2 gene expression)+1.988*(IG/LPS induced CCL2 gene expression ratio)−0.684*(Proportion of T-cells)+1.901,
wherein values higher than the maximal Youden Index indicate responders and are awarded a “0” for the Responder Score calculation, while values below indicate non-responders and are awarded a “1”.

16. The method of claim 1 wherein the individual LDA-score related to the COPS8 genotype is calculated by the formula:
Individual COPS8-LDA-score=−6.442*(COPS8 genotype result)+1.592*(IG/LPS induced CXCL8 gene expression)+0.911*(IG/LPS induced(netto)CCL2 gene expression)+1.930*(IG/LPS induced(netto)IL-1β gene expression)+3.245,
wherein values higher than the maximal Youden Index indicate responders and are awarded a “0” for the Responder Score calculation, while values below indicate non-responders and are awarded a “1”.

17. The method of claim 1 wherein the individual LDA-score related to the ADAMTSL1 genotype is calculated by the formula:
Individual ADAMTSL1-LDA-score=3.369*(ADAMTSL1 genotype result)−1.897*(LPS induced CXCL2 gene expression)+1.212*(LPS induced CXCL10 gene expression)+1.152*(LPS stimulated IL-1β gene expression)+0.859*(IG/LPS induced(netto)CXCL2 gene expression)−1.171,
wherein values lower than the maximal Youden Index indicate responders and are awarded a “0” for the Responder Score calculation, while values above indicate non-responders and are awarded a “1”.

18. The method of claim 1 wherein the individual LDA-score related to the CXXC4 genotype is calculated by the formula:
Individual CXXC4-LDA-score=2.925*(CXXC4 genotype result)+1.992*(IG/LPS induced (netto)IFN-γ gene expression)−1.099*(IG induced(netto)CXCL9 protein release)−1.476,
wherein values lower than the maximal Youden Index indicate responders and are awarded a “0” for the Responder Score calculation, while values above indicate non-responders and are awarded a “1”.

19. The method of claim 1 wherein the individual LDA-score related to the RSPO2 genotype is calculated by the formula:
Individual RSPO2-LDA-score=2.975*(RSPO2 genotype result)+0.7941*(sICAM1 plasma concentration)−1.491,
wherein values lower than the maximal Youden Index indicate responders and are awarded a “0” for the Responder Score calculation, while values above indicate non-responders and are awarded a “1”.

20. The method of claim 1 wherein 2 to 9 results of individual LDA-scores are combined, by addition, to create the Responder Score and wherein the cut-off to discriminate between responders and non-responders is set by adding 4 times the standard deviation of the reference data to the highest obtained score in the reference responder group.

21. The method of claim 20 wherein 9 results of individual LDA-scores are combined, by addition, to create the Responder Score.

22. The method of claim 1 wherein the immune parameters are determined after ex vivo exposure of samples with immunoglobulin.

23. The method of claim 1 wherein genotyping and immune parameters are determined in whole blood, blood fractions, cell fractions or plasma.

24. The method according to claim 1 wherein the amount of immunoglobulins used in assays is from about 0.01 to about 100 mg/ml.

25. The method of claim 1 wherein the method is performed before and/or during the treatment of a patient with immunoglobulin.

26. The method of claim 1 wherein any immunoglobulin product suitable for in vivo use is concerned.

27. A method for determining the likelihood of response of a patient, suffering from a disease, towards immunoglobulin therapy comprising the steps of
a) providing a sample of the patient containing B- and T-lymphocytes, natural killer cells, invariant T-cells and monocytes;
b) determination of values of immune parameters which are either static, such as leukocyte subpopulations and cytokine-level in the plasma or functional, like gene expression and cytokine release after lipopolysaccharide (LPS) and/or IVIG stimulation ex vivo;
c) genotyping of at least two of the polynucleotides selected from the group consisting of MTM1, EIF3E, COPSE, ADAMTSL1, CXXC4, RSPO2, OR9Q1, ADAMTS9, KLHDC8A and PRDM9, and awarding the value of 0 for specific homozygous SNP combinations (SNP—Single Nucleotide Polymorphism);
d) combining the results of genotyping of step c) with results of immuno parameter determination of step b) to calculate a the patient's LDA-score (LDA—Linear Discriminant Analysis);
e) combining the patient's LDA-scores by addition to obtain a patient's Responder score;
f) comparing said patient's Responder score with the Responder score of the reference generated by the method according to at least one of the claim 1, wherein the cut-off to discriminate between responders and non-responders is set by adding 4 times the standard deviation of the reference data to the highest obtained score in the reference responder group;
g) and a patient's Responder score below this cut-off indicates a responder, whereas a patient's Responder score above this cut-off indicates a non-responder.

28. The method according to at least one of the claim 1 wherein the disease is selected from the group consisting of inflammatory mediated immune diseases, autoimmune diseases, allergies, graft-versus-host reactions and prevention of transplant rejection; any kind of multiple sclerosis or any other demyelinating neurological disease; and relapsing-remitting multiple sclerosis.

29. The method according to claim 1 to predict the probability of a relapse of a MS patient and/or the rate of progression of the disease in terms of disability and or functioning of the patient as measured by a clinical scale.

30. The method of claim 1 which facilitates the approval or recommendation of immunoglobulins by health authorities for the treatment of any kind of multiple sclerosis or any other demyelinating disease or Lupus erythematosus, rheumatoid arthritis or intestinal/bowel diseases such as Crohn's disease, myositis or recurrent abortion.

31. The method of claim 1, wherein
(a) samples are provided from at least 10 individuals and/or
(b) the static immune parameters are leukocyte subpopulations or cytokine-level in the plasma; and/or the functional immune parameters are gene expression and cytokine release after lipopolysaccharide (LPS) and/or IVIG stimulation ex vivo.

32. The method of claim 1, wherein
the samples are exposed to the immunoglobulin IgG, IgM, IgA or a combination thereof and/or
the amount of immunoglobulins used in the assays is from about 1 to about 50 mg/ml and/or
the immununoglobulin product is applied intravenously, subcutaneously, intramuscularly, ocularly, intrathecially, orally, topically or inhalably.

33. The method of claim 29, wherein the clinical scale which is measured comprises the expanded disability status scale (EDSS).

34. The method of claim 33, wherein the EDSS is lupus erythematosis, rheumatoid arthritis, or the intestinal/bowel disease Crohn's disease, myositis or recurrent abortion.
